SEQUENCE LISTING (16 sequences)

SEQ ID NO: 1 - DNA, 21 bp, synthetic construct
  source 1..21: mol_type = other DNA
caagaaggctgttgctgttcg

SEQ ID NO: 2 - DNA, 20 bp, synthetic construct
  source 1..20: mol_type = other DNA
ggcagaagctgtcgatgatt

SEQ ID NO: 3 - DNA, 22 bp, synthetic construct
  source 1..22: mol_type = other DNA
catactcctgggtctggttggt

SEQ ID NO: 4 - DNA, 20 bp, synthetic construct
  source 1..20: mol_type = other DNA
gacagccatccgcatcttct

SEQ ID NO: 5 - DNA, 20 bp, synthetic construct
  source 1..20: mol_type = other DNA
cctgctcaccctcattggag

SEQ ID NO: 6 - DNA, 20 bp, synthetic construct
  source 1..20: mol_type = other DNA
gctgaactcactcttgggct

SEQ ID NO: 7 - DNA, 20 bp, synthetic construct
  source 1..20: mol_type = other DNA
tgtagccacagcaagaggtg

SEQ ID NO: 8 - DNA, 20 bp, synthetic construct
  source 1..20: mol_type = other DNA
ctggaatggctccttgtggt

SEQ ID NO: 9 - DNA, 19 bp, synthetic construct
  source 1..19: mol_type = other DNA
ccgtaaccccgagttggat

SEQ ID NO: 10 - DNA, 19 bp, synthetic construct
  source 1..19: mol_type = other DNA
attgaggcggtcgttgatg

SEQ ID NO: 11 - DNA, 20 bp, synthetic construct
  source 1..20: mol_type = other DNA
gaagcgctgaaccgatacca

SEQ ID NO: 12 - DNA, 21 bp, synthetic construct
  source 1..21: mol_type = other DNA
tgcttctgtgcctcagtttcc

SEQ ID NO: 13 - DNA, 20 bp, synthetic construct
  source 1..20: mol_type = other DNA
cctctctggcaaagtccaag

SEQ ID NO: 14 - DNA, 20 bp, synthetic construct
  source 1..20: mol_type = other DNA
ggtcacgctcctggaagata

SEQ ID NO: 15 - DNA, 20 bp, synthetic construct
  source 1..20: mol_type = other DNA
gactgctgctgacaccttca

SEQ ID NO: 16 - DNA, 20 bp, synthetic construct
  source 1..20: mol_type = other DNA
accggactgttaccaacacc

(19) United States
(12) Patent Application Publication
(10) Pub. No.: US 2026/0217802 A1
(43) Pub. Date: Jul. 30, 2026

(54) ANIMAL FEED SUPPLEMENT

(71) Applicant: The United States of America, as Represented by the Secretary of Agriculture, Washington, DC (US)

(72) Inventors: REVATHI SHANMUGASUNDARAM, BOGART, GA (US); PRASANNA H. GOWDA, STONEVILLE, MS (US)

(21) Appl. No.: 19/039,990

(22) Filed: Jan. 29, 2025

(51) Int. Cl. C07K 16/14; A23K 20/147; A61K 9/00; A61K 39/00; A61P 39/02

(52) U.S. Cl. CPC C07K 16/14; A23K 20/147; A61K 9/0053; A61P 39/02; A61K 2039/505; C07K 2317/11; C07K 2317/76

(57) ABSTRACT

Disclosed are nanoparticle-mycotoxin-hemocyanin complexes useful in the prevention or amelioration of symptoms associated with mycotoxicosis in an animal in need thereof. Disclosed are methods for preparing such complexes, and use of such complexes in vaccinating animals in need thereof to prevent or ameliorate symptoms associated with mycotoxicosis. Disclosed are egg compositions enriched in at least one anti-mycotoxin antibody, feed supplements and feed comprising such egg compositions, and methods for the preparation of such egg compositions, feed supplements, and feed. Disclosed are methods of using such egg compositions, feed supplements, or feed to prevent or ameliorate symptoms associated with mycotoxicosis in animals fed mycotoxin-contaminated feed.

### FIELD

The disclosure relates to egg compositions enriched in at least one anti-mycotoxin antibody, feed supplements and feed comprising such egg compositions, and methods for the preparation of such egg compositions, feed supplements, and feed. Disclosed are methods of using such egg compositions, feed supplements, or feed to prevent or ameliorate symptoms associated with mycotoxicosis in animals fed mycotoxin-contaminated feed.

### SEQUENCE LISTING

The instant application contains a Sequence Listing XML required by 37 C.F.R. § 1.831(a) which has been submitted in XML file format via the USPTO patent electronic filing system, and is hereby incorporated by reference in its entirety. The XML file was created on 01/27/2025, is named Sequence_Listing-0041.24, and has 15 KB.

### BACKGROUND

Mycotoxins are secondary metabolites secreted by a variety of fungi, often produced in cereal grains as well as forages before, during, and/or after harvest. Forages and cereals naturally come into contact with fungal spores. The fungal contamination of plants and the bio-synthesis of toxins depend on the state of health of the plant before harvest, meteorological conditions, harvesting techniques, delays and hydrothermal conditions before stabilization for conservation and feed processing. Depending on the fungus, fungal growth is controlled by a number of physicochemical parameters including the amount of free water, temperature, presence of oxygen, nature of the substrate, and pH conditions. Mycotoxins proliferate pre-harvest as well as post-harvest in storage.

The effects of mycotoxins vary greatly in their severity. Some mycotoxins are lethal, some cause identifiable diseases or health problems, some weaken the immune system without producing symptoms specific to a mycotoxin, some act as allergens or irritants, and some have no known effect on animals or humans. Mycotoxin contamination in feed is a significant problem that adversely affects animal production, especially in poultry and swine. The mycotoxins often influence gut integrity and liver function, and ultimately impact animal performance. The worldwide economic impact for the US$40 billion poultry industry is upwards of US$2 billion.

Many animals are susceptible to hundreds of mycotoxins. Pathogenicity in is dependent on the life stage and species of the animal affected as well as the level of mycotoxin exposure and individual susceptibility. Mycotoxins can have additive, synergistic, and antagonistic effects with other toxins, infectious agents, and nutritional requirements. Mycotoxins can also interact with drugs used as therapeutic agents and diminish the effects of vaccines administered to animals such as fish, birds, reptiles, amphibians, or mammals.

Common mycotoxins associated with mycotoxicosis in include aflatoxins, ochratoxins, fumonisins, zearalenone, and trichothecenes. Common trichothecenes are deoxynivalenol (DON; also known as vomitoxin) and T-2 mycotoxins.

For treatment of mycotoxicosis in domestic animals, it is suggested that contaminated feed or bedding should be replaced, and concurrent diseases should be treated to alleviate disease interactions. Nonspecific treatments using activated charcoal (digestive tract adsorption) in the feed have a sparing effect but are not practical for larger production units. In larger scale production, the use of clays and derivatives from yeast, bacteria, and plants has more practical applications. Certain enzymes have potential to biodegrade specific mycotoxins, such as fumonisins, into nontoxic metabolites (enzymatic biotransformation). However, availability of detoxifying enzymes is limited. Off-label treatment with antifungal drugs labeled for use in other species (eg, nystatin and triazole antifungal agents) may not be permitted in certain jurisdictions.

Thus, a need exists for the development of new non-antibiotic strategies for the control and/or prevention of mycotoxicosis.

### SUMMARY

Provided herein are nanoparticle-based anti-mycotoxin vaccines, methods of preparing such vaccines, and methods of using such vaccines to control and/or prevent mycotoxicosis. Included are compositions comprising egg enriched in at least one anti-mycotoxin antibody, methods for preparing such compositions, and use of such compositions for protecting animals from mycotoxin infection.

In an embodiment, the disclosure relates to an immunogenic composition comprising at least one mycotoxin bound to a carrier protein and entrapped within a nanoparticle. In some embodiments of the disclosure, the at least one mycotoxin is an aflatoxin, an ochratoxin, a fumonisin, a zearalenone, a trichothecene, or a mixture thereof. In some embodiments of the disclosure the carrier protein is a hemocyanin. In some embodiments of the disclosure the carrier protein is Keyhole limpet hemocyanin (KLH). In some embodiments of the disclosure the nanoparticle comprises a chitosan.

In an embodiment, the disclosure relates to a method for treating an animal in need thereof to prevent or ameliorate one or more symptoms of mycotoxicosis, the method comprising administering to the animal at least one effective dose of a composition comprising at least one mycotoxin bound to a carrier protein and entrapped within a nanoparticle. In some embodiments of the disclosure, the method protects the animal from one or more clinical signs of mycotoxin-induced mycotoxicosis. In some embodiments of the disclosure, the treated animal is a fish, a bird, a reptile, an amphibian, or a mammal.

In an embodiment, the disclosure relates to a method for preparing a composition comprising at least one mycotoxin bound to a carrier protein and entrapped within a nanoparticle. The method comprises linking at least one mycotoxin to a carrier protein to prepare a mycotoxin-carrier protein complex; adding drop by drop the mycotoxin-carrier protein complex into a nanoparticle solution and stirring for a determined amount of time; adding sodium tripolyphosphate as a linking agent; and collecting nanoparticle-mycotoxin-carrier protein complex; where the mycotoxin is bound to the carrier protein and entrapped within the nanoparticle.

In an embodiment, the disclosure relates to a composition comprising an egg or a part thereof enriched in at least one anti-mycotoxin antibody. In some embodiments of the disclosure the egg or part thereof enriched in at least one anti-mycotoxin antibody in the composition is from an anti-mycotoxin-vaccinated egg-producing animal.

In an embodiment, the disclosure relates to a feed supplement comprising egg or a part thereof enriched in at least one anti-mycotoxin antibody. In some embodiments of the disclosure, the feed supplement comprising an egg or a part thereof enriched in at least one anti-mycotoxin antibody is for administration to a fish, a bird, a reptile, an amphibian, or a mammal. In some embodiments of the disclosure the bird is a domesticated or wild fowl bird. In some embodiments of the disclosure, the mammal is a wild or domesticated mammal.

In an embodiment, the disclosure relates to a feed comprising feed material and a feed supplement comprising an egg or a part thereof enriched in at least one anti-mycotoxin antibody. In some embodiments of the disclosure the feed comprising feed material and a feed supplement comprising an egg or a part thereof enriched in at least one anti-mycotoxin antibody further comprises at least one additive. In some embodiments of the disclosure, the feed comprising feed material and a feed supplement comprising an egg or a part thereof enriched in at least one anti-mycotoxin antibody is for administration to birds, fish, reptiles, or mammals.

In an embodiment, the disclosure relates to a method for preparing a composition comprising egg or a part thereof enriched in at least one anti-mycotoxin antibody, the method comprising collecting eggs laid by an egg-producing animal vaccinated with at least one anti-mycotoxin vaccine; optionally separating egg yolks or egg whites from the collected eggs; and drying the collected eggs, egg yolks, egg whites, or a mixture thereof.

In an embodiment, the disclosure relates to a method of preventing or ameliorating one or more symptoms of mycotoxicosis in an animal in need thereof, the method comprising administering to the animal at least one dose of a composition comprising egg or a portion thereof enriched in at least one anti-mycotoxin antibody, wherein administering the composition to the animal protects the animal from one or more clinical signs of mycotoxin-induced mycotoxicosis. In some embodiments of the disclosure, the composition comprising egg or a portion thereof enriched in at least one anti-mycotoxin antibody is provided to the animal in feed or drinking water.

### BRIEF DESCRIPTION OF THE DRAWINGS

These and other features, aspects, and advantages of the present disclosure will become better understood with regard to the following description, appended claims, and accompanying drawings.

FIG. 1 and FIG. 1B depict the chemical structures of fumonosin B1 (FUM) and deoxynivalenol (DON). FIG. 1 shows the chemical structure of FUM. FIG. 1B shows the chemical structure of DON.

FIG. 2A and FIG. 2B depict a schematic diagram of the preparation of a vaccine comprising at least one mycotoxin bound to a carrier protein and entrapped within a nanoparticle, and a vaccine particle. FIG. 2A shows a schematic diagram of the preparation of a vaccine comprising at least one mycotoxin bound to a carrier protein and entrapped within a nanoparticle. FIG. 2B shows a schematic diagram of a mycotoxin-carrier protein-nanoparticle complex.

FIG. 3 depicts a graph of the anti-FUM IgY and anti-DON IgY in serum from layer birds vaccinated with CS-FUM-DON-KLH. Negative control is shown as a black bar; anti-FUM IgY is shown as a striped bar; anti-DON IgY is shown as a gray bar. The Y axis shows the absorbance value at 450 nm. The X axis shows the collection days. Days post vaccination=dpv.

FIG. 4 depicts a graph of the body weight of layer chickens prior to vaccination and 7 days post vaccination (dpv) with CS-FUM-DON-KLH. The Y axis shows the body weight in kilograms. The X axis shows the collection days.

FIG. 5A and FIG. 5B depict graphs of the anti-FUM IgY and anti-DON IgY in serum as measured by ELISA. FIG. 5A shows the anti-FUM IgY. FIG. 5B shows the anti-DON IgY. The Y Axis shows the measured OD values at 450 nm. The X Axis shows the treatments: not-vaccinated animals feed regular diet (CON); not-vaccinated animals fed diet supplemented with FUM and DON (FUM+DON); vaccinated animals fed regular diet (Vaccine); vaccinated animals fed diet supplemented with FUM and DON (FUM+DON+Vaccine). Values with no common letter differ significantly (P≤0.05).

FIG. 6 depicts a graph of the body weight of layer chickens after 21 days. The Y axis shows the chicken weight in grams. The X axis shows the treatments: non-vaccinated chickens fed regular diet (Control); non-vaccinated chickens fed diet supplemented with FUM and DON (FUM+DON); chickens fed regular diet and vaccinated with CS-FUM-DON-KLH (Vaccine); chickens fed diet supplemented with FUM and DON and vaccinated with CS-FUM-DON-KLH (FUM+DON+Vaccine). Values with no common letter differ significantly (P<0.05).

FIG. 7 depicts a 21 day feed consumption graph. The Y axis shows the amount of feed consumption in grams (g). The X axis shows the treatments: non-vaccinated chickens fed regular diet (Control); non-vaccinated chickens fed diet supplemented with FUM and DON (FUM+DON); chickens fed regular diet and vaccinated with CS-FUM-KLH and CS-DON-KLH (Vaccine); chickens fed diet supplemented with FUM and DON and vaccinated with CS-FUM-KLH and CS-DON-KLH (FUM+DON+Vaccine).

FIG. 8 depicts a graph of the feed conversion ration (FCR) measured at day 21 post vaccination. The Y axis shows the FCR. The X axis shows the treatments: non-vaccinated chickens fed regular diet (Control); non-vaccinated chickens fed diet supplemented with FUM and DON (FUM+DON); chickens fed regular diet and vaccinated with CS-FUM-KLH and CS-DON-KLH (Vaccine); chickens fed diet supplemented with FUM and DON and vaccinated with CS-FUM-KLH and CS-DON-KLH (FUM+DON+Vaccine). Values with no common letter differ significantly (P<0.05).

FIG. 9 depicts a graph of the day 21 serum fluorescein isothiocyanate dextran (FITC-d) as an indication of inflammation. The Y axis shows the serum FITC-D in μg/mL. The X axis shows the treatments: non-vaccinated chickens fed regular diet (Control); non-vaccinated chickens fed diet supplemented with FUM and DON (FUM+DON); chickens fed regular diet and vaccinated with CS-FUM-KLH and CS-DON-KLH (Vaccine); chickens fed diet supplemented with FUM and DON and vaccinated with CS-FUM-KLH and CS-DON-KLH (FUM+DON+Vaccine). Values with no common letter differ significantly (P<0.05).

FIG. 10A and FIG. 10B depict graphs of the day 21 anti-DON and anti-FUM bile IgA. FIG. 10A shows the results for anti-DON IgA. FIG. 10B shows the results for anti-FUM IgA. The Y axis shows the OD_{450 nm}. The X axis shows the treatments: non-vaccinated chickens fed regular diet (Control); non-vaccinated chickens fed diet supplemented with FUM and DON (FUM+DON); chickens fed regular diet and vaccinated with CS-FUM-KLH and CS-DON-KLH (Vaccine); chickens fed diet supplemented with FUM and DON and vaccinated with CS-FUM-KLH and CS-DON-KLH (FUM+DON+Vaccine). Values with no common letter differ significantly (P≤0.05).

FIG. 11A and FIG. 11B depict graphs of the anti-FUM and anti-DON IgG in day 21 serum. FIG. 11A shows data for anti-FUM IgG. FIG. 11B shows data for anti-DON IgG. The Y axis shows the OD_{450 nm}. The X axis shows the treatments: non-vaccinated chickens fed regular diet (Control); non-vaccinated chickens fed diet supplemented with FUM and DON (FUM+DON); chickens fed regular diet and vaccinated with CS-FUM-KLH and CS-DON-KLH (Vaccine); chickens fed diet supplemented with FUM and DON and vaccinated with CS-FUM-KLH and CS-DON-KLH (FUM+DON+Vaccine). Values with no common letter differ significantly (P≤0.05).

FIG. 12 depict images of bird liver sections obtained from birds under different treatments. not vaccinated birds fed regular diet (Control); not vaccinated birds fed diet supplemented with FUM and DON (FUM+DON); vaccinated birds fed regular diet (Vaccine); vaccinated birds fed diet supplemented with FUM and DON (FUM+DON+Vaccine). Arrows show mononuclear cell infiltration (MNCI), fibrosis and bile duct proliferation (F+BDP), and hepatocyte hydropic degeneration (HHD).

FIG. 13 depicts a graph of the FUM-DON-specific memory lymphocytes induced by the nanoparticle vaccine in a recall assay at day 21. The Y Axis shows the OD_{570 nm}. The X axis shows the treatments: Negative control, lymphocytes isolated from non-vaccinated chickens fed regular diet (Con); positive control, lymphocytes isolated from chickens vaccinated with FUM-DON-KLH antigen and fed regular diet (Con+Ag); lymphocytes from chickens fed diet supplemented with egg powder laid by chickens (Egg Powder+Ag); lymphocytes from chickens vaccinated with FUM-DON-KLH antigen and fed diet contaminated with FUM+DON (MTX+Ag); lymphocytes from chickens fed diet contaminated with FUM+DON and supplemented with egg powder from chickens vaccinated with FUM-DON-KLH antigen (MTX+Egg Powder+Ag). Values with no common letter differ significantly (P<0.05).

FIG. 14A and FIG. 14B depict graphs of the serum aspartate aminotransferase and creatine kinase detected at day 21 after different treatments. FIG. 14A shows serum aspartate aminotransferase data. FIG. 14B shows creatine kinase data. The Y axis shows the enzyme levels in units per liter (AST U/L). The X axis shows the treatments: non-vaccinated chickens fed regular diet (Control); non-vaccinated chickens fed diet supplemented with FUM and DON (FUM+DON); chickens fed regular diet and vaccinated with CS-FUM-KLH and CS-DON-KLH (Vaccine); chickens fed diet supplemented with FUM and DON and vaccinated with CS-FUM-KLH and CS-DON-KLH (FUM+DON+Vaccine). Values with no common letter differ significantly (P<0.05).

FIG. 15 depicts a graph of the IgY detected in layer chicken serum at 4, 5, 6, 7 8, or 9 days post-booster after vaccination with CS-FUM-KLH and CS-DON-KLH. The Y axis shows the OD450 value. The X axis shows the sample day. Dark grey bars show data for non-vaccinated chickens (Negative Ctrl); light grey bars show anti FUM IgY; black bars show anti DON IgY.

FIG. 16 depicts a graph of the IgY detected in yolks of eggs laid 4, 5, 6, 7 8, or 9 days post-booster with CS-FUM-KLH and CS-DON-KLH. The Y axis shows the OD 450 value. The X axis shows the sample day. Dark grey bars show data for non-vaccinated chickens (Negative Ctrl); light grey bars show anti FUM IgY; black bars show anti DON IgY.

FIG. 17 depicts graphs to determine the lowest dilution of egg yolk antibodies that recognize FUM using ELISA in 4 different egg yolks samples from eggs laid 5 days post-booster vaccination. The Y axis shows the OD 450 value. The X axis shows the egg yolk powder concentration in μg/mL.

FIG. 18A to FIG. 18D depict graphs of the fate of HD11 cells under different treatments. FIG. 18A shows the effect of adding media supplemented with FUM. FIG. 18B shows effect of adding media supplemented with FUM and egg yolk powder obtained from eggs laid by anti-FUM/DON vaccinated chickens. FIG. 18C shows effect of adding media supplemented with DON. FIG. 18D shows effect of adding media supplemented with DON and egg yolk powder obtained from eggs laid by anti-FUM/DON vaccinated chickens.

FIG. 19 depicts a graph of survival of HD11 cells under different treatments. The Y axis shows the percentage of dead cells. The X axis shows the different assayed groups: annexin positive (early apoptosis); 7-AAD positive (late apoptosis); and total apoptotic cells. Each group shows the results obtained after treating the cells with either regular media (control); regular media supplemented with egg yolk powder from anti-FUM/DON vaccinated chickens (control+AB); regular media supplemented with FUM (FUM); regular media supplemented with FUM and egg yolk powder from anti-FUM/DON vaccinated chickens (FUM+AB); regular media supplemented with DON (DON); regular media supplemented with DON and egg yolk powder from anti-FUM/DON vaccinated chickens (DON+AB); regular media supplemented with FUM and DON (FUM+DON); regular media supplemented with FUM, DON, and egg yolk powder from anti-FUM/DON vaccinated chickens (FUM+DON+AB).

FIG. 20 depicts a graph of the effect of FUM and DON on NO production in HD11 cells under different treatments. The Y axis shows the nitrite concentration in μM. The X axis presents the different cell treatments: regular cell media (Control); cell media supplemented with FUM (FUM); cell media supplemented with DON (DON); cell media supplemented with FUM and DON (FUM+DON); cell media supplemented with FUM and egg yolk powder from anti-FUM/DON-vaccinated chickens (FUM+AB); cell media supplemented with DON and egg yolk powder from anti-FUM/DON-vaccinated chickens (DON+AB); cell media supplemented with FUM, DON, and egg yolk powder from anti-FUM/DON vaccinated chickens (FUM+DON+AB).

FIG. 21 depicts a graph of the d21 body weight of layer chickens after different treatments. The Y axis shows the body weight in grams (g). The X axis shows the treatments: chickens fed regular diet (control); chickens fed diet supplemented with FUM and DON (FUM+DON); chickens fed diet supplemented with egg powder of the instant disclosure (EggPowder); chickens fed diet supplemented with FUM, DON, and egg powder of the instant disclosure (FUM+DON+EggPowder). Values with no common letter differ significantly (P<0.05).

FIG. 22 depicts a graph of layer chickens' food consumption under different treatments. The Y axis shows the food consumption in grams (g). The X axis shows the treatments: chickens fed regular diet (control); chickens fed diet supplemented with FUM and DON (FUM+DON); chickens feed diet supplemented with egg powder of the instant disclosure (EggPowder); chickens fed diet supplemented with FUM, DON, and egg powder of the instant disclosure (FUM+DON+EggPowder).

FIG. 23 depicts a graph of the feed conversion ratio (FCR) of layer chickens under different treatments. The Y axis shows the FCR. The X axis shows the treatments. chickens fed regular diet (Control); chickens fed diet supplemented with FUM and DON (FUM+DON); chickens fed diet supplemented with egg powder of the instant disclosure (EggPowder); chickens fed diet supplemented with FUM, DON, and egg powder of the instant disclosure (FUM+DON+EggPowder). Values with no common letter differ significantly (P<0.05).

FIG. 24 depicts images of chicken livers from layer chickens after different treatments. Treatments are shown on the bottom of each photograph: chicken fed regular diet (Control); chicken fed diet supplemented with FUM and DON (FUM+DON); chicken fed diet supplemented with egg powder of the instant disclosure (Egg Powder); chicken fed diet supplemented with FUM, DON, and egg powder of the instant disclosure (FUM+DON+Egg Powder).

FIG. 25 depicts a graph of the serum FITC-d at d21 as an indication of inflammation in layer chickens. The Y axis shows the serum FITC-d (g/mL). The X axis shows the treatments: chickens fed regular diet (Control); chickens fed diet supplemented with FUM and DON (FUM+DON); chickens fed diet supplemented with egg powder of the instant disclosure (EggPowder); chickens fed diet supplemented with FUM, DON, and egg powder of the instant disclosure (FUM+DON+EggPowder). Values with no common letter differ significantly (P<0.05).

FIG. 26 depicts a graph of the d21 bile anti-DON IgA in layer chickens. The Y axis shows the OD value at 450 nm. The X axis shows the treatments: chickens fed regular diet (Control); chickens fed diet supplemented with FUM and DON (FUM+DON); chickens fed diet supplemented with egg powder of the instant disclosure (EggPowder); chickens fed diet supplemented with FUM, DON, and egg powder of the instant disclosure (FUM+DON+EggPowder).

FIG. 27 depicts images of chicken jejunal villi after different treatments. The treatments are indicated below the photographs. Chicken fed regular diet (Control); chicken fed diet supplemented with FUM and DON (FUM+DON); chicken fed diet supplemented with egg powder of the instant disclosure (EggPowder); chickens fed diet supplemented with FUM, DON, and egg powder of the instant disclosure (FUM+DON+EggPowder).

FIG. 28 depicts images of liver sections from birds fed different diets. The treatments are indicated below the photographs: birds fed regular diet (Control); birds fed diet supplemented with FUM and DON (FUM+DON): birds fed diet supplemented egg yolk powder containing anti-FUM and anti-DON IgY (Egg Powder); birds fed diet supplemented with FUM, DON, and egg yolk powder containing anti-FUM and anti-DON IgY (FUM+DON+Egg Powder). Arrows show mononuclear cell infiltration (MNCI), fibrosis and bile duct proliferation (F+BDP), and hepatocyte hydropic degeneration (HHD).

FIG. 29A and FIG. 29B depict graphs of the serum aspartate aminotransferase and creatine kinase detected in layer chickens at day 21 after different treatments. FIG. 29A shows serum aspartate aminotransferase data. FIG. 29B shows creatine kinase data. The Y axis shows the enzyme levels in units per liter (U/L). The X axis shows the treatments: chickens fed regular diet (Control); chickens fed diet supplemented with FUM and DON (FUM+DON); chickens fed diet supplemented egg yolk powder containing anti-FUM and anti-DON IgY (Egg Powder); chickens fed diet supplemented with FUM, DON, and egg yolk powder containing anti-FUM and anti-DON IgY (FUM+DON+Egg Powder). Values with no common letter differ significantly (P≤0.05).

### BRIEF DESCRIPTION OF THE SEQUENCE LISTING

The nucleotide sequences disclosed in the disclosure are listed in Table 1 below, including their sequence identifier and type of sequence.

TABLE 1
Sequences

| Identifier | Type | Primer Name | Primer Sequence (5'-3') |
|---|---|---|---|
| SEQ ID NO: 01 | DNA | RPS 13 forward | CAAGAAGGCTGTTGCTGTTCG |
| SEQ ID NO: 02 | DNA | RPS 13 reverse | GGCAGAAGCTGTCGATGATT |
| SEQ ID NO: 03 | DNA | Claudin-1 forward | CATACTCCTGGGTCTGGTTGGT |
| SEQ ID NO: 04 | DNA | Claudin-1 reverse | GACAGCCATCCGCATCTTCT |
| SEQ ID NO: 05 | DNA | Claudin-2 forward | CCTGCTCACCCTCATTGGAG |
| SEQ ID NO: 06 | DNA | Claudin-2 reverse | GCTGAACTCACTCTTGGGCT |
| SEQ ID NO: 07 | DNA | Z. Occluden-1 forward | TGTAGCCACAGCAAGAGGTG |
| SEQ ID NO: 08 | DNA | Z. Occluden-1 reverse | CTGGAATGGCTCCTTGTGGT |
| SEQ ID NO: 09 | DNA | Occludin forward | CCGTAACCCCGAGTTGGAT |
| SEQ ID NO: 10 | DNA | Occludin reverse | ATTGAGGCGGTCGTTGATG |
| SEQ ID NO: 11 | DNA | Claudin-4 forward | GAAGCGCTGAACCGATACCA |
| SEQ ID NO: 12 | DNA | Claudin-4 reverse | TGCTTCTGTGCCTCAGTTTCC |
| SEQ ID NO: 13 | DNA | GAPDH forward | CCTCTCTGGCAAAGTCCAAG |
| SEQ ID NO: 14 | DNA | GAPDH reverse | GGTCACGCTCCTGGAAGATA |
| SEQ ID NO: 15 | DNA | β-actin forward | GACTGCTGCTGACACCTTCA |
| SEQ ID NO: 16 | DNA | β-actin reverse | ACCGGACTGTTACCAACACC |

### DETAILED DESCRIPTION

The disclosure relates to immunogenic compositions comprising at least one mycotoxin bound to a carrier protein and entrapped within a nanoparticle. The compositions are useful in methods for of treating an animal in need thereof to prevent or ameliorate one or more symptoms of mycotoxicosis, the method comprising administering to the animal at least one effective dose of the disclosed vaccine composition. Disclosed are compositions comprising an egg or part thereof enriched in at least one anti-mycotoxin antibody. The egg or part thereof in the composition may be from an anti-mycotoxin-vaccinated egg-producing animal. The compositions are useful as a feed supplement in preventing or ameliorating one or more symptoms of mycotoxicosis in susceptible animals.

Unless otherwise explained, all technical and scientific terms used herein have the same meaning as commonly understood by one of ordinary skill in the art to which this disclosure belongs.

As used herein, the singular terms “a”, “an”, and “the” include plural referents unless context clearly indicates otherwise. Similarly, the word “or” is intended to include “and” unless the context clearly indicate otherwise.

Other than in the operating examples, or where otherwise indicated, all numbers expressing quantities of ingredients, concentrations, or reaction conditions used herein should be understood as modified in all instances by the term “about.”

As used herein, the term “about” is defined as plus or minus ten percent of a recited value. For example, about 1.0 g means 0.9 g to 1.1 g.

As used in the disclosure, the term “egg-producing animal” means any oviparous animal, and includes any animal that lays an egg, such as birds, fish, and reptiles.

As used herein, the term “bird” refers to an animal that is a member of the class Aves. Birds include, but are not limited to, domesticated and wild fowl. Domesticated fowl are birds raised for their meat, eggs, or feathers. Domesticated and wild fowl include chickens, turkeys, geese, ducks, pheasants, quail, pigeons, and ostriches.

As used herein, the term “enriched” refers to an egg or part thereof having anti-mycotoxin antibody levels higher than the levels of anti-mycotoxin antibody found in an egg or part thereof of an egg produced by an egg-producing animal not vaccinated or treated with an anti-mycotoxin antibody.

As used herein, the term “hyperimmunization” means exposure to one or more antigens such that an immune response is elevated and maintained above the natural unexposed state.

The term “egg” as used herein refers to a whole egg or part thereof. The term “egg product” as used herein refers to a whole egg or any product or fraction obtained from a whole egg. The egg product may be an egg yolk, an egg white, or a whole egg. The egg yolk may be in the form of an egg yolk powder, the egg white may be in the form of egg white powder, and the whole egg may be in the form of a whole egg powder.

The term “hyperimmunized egg” refers to an egg obtained from an egg-producing animal maintained in a hyperimmune state, i.e. an egg-producing animal that has been vaccinated with a mycotoxin antigen. The term “hyperimmunized egg product” refers to a hyperimmunized egg or any product obtained from a hyperimmunized egg.

As used herein the term “concentrate” refers to a hyperimmunized egg product that is at least partially purified, such that the concentration of antibodies in the concentrate is greater than the concentration of antibodies in a hyperimmunized egg. The hyperimmunized egg product may be a concentrate.

The term “aqueous IgY concentrate” as used herein refers to an aqueous solution comprising IgY antibodies obtained from a hyperimmunized egg. The concentration of IgY antibodies in the aqueous solution is higher than the concentration of antibodies in the hyperimmunized egg. The hyperimmunized egg product may be an aqueous IgY concentrate.

In the instant disclosure, the term “egg powder” refers to powder obtained from egg or part thereof that has been dried. The egg powder may be a “whole egg powder” where the powder is obtained from an egg; an “egg yolk powder” where the powder is obtained from egg yolk; an “egg white powder” where the powder is obtained from egg yolk.

The egg powder, egg yolk powder, or egg white powder may be prepared by any means known in the art. The egg powder, egg yolk powder, or egg white powder may be dried by spray drying, milling (including jet milling), freeze-drying (or spray freeze-drying), supercritical fluid drying, solution-mediated crystallization, in a continuous fluid-bed system, and thermal decomposition.

The term “supranormal levels” means levels in excess of those found in eggs of egg-producing animals that are not hyperimmunized. For example, supranormal levels of an antibody to a particular antigen are levels of the antibody in excess of those found in eggs of egg-producing animals that are not immunized with the particular antigen.

As used herein, a “feed supplement” is a combination of nutrients or compounds that are added to animal feed to improve the nutritional value of the feed and the health of the animals.

Mention of trade names or commercial products in this disclosure is solely for the purpose of providing specific information and does not imply recommendation or endorsement of the trade name or commercial product.

Mycotoxins are naturally-occurring toxic chemical compounds produced by fungi that readily colonize crops. Fungi that can produce mycotoxins grow on numerous foodstuffs such as cereals, dried fruits, nuts and spices. Fungal growth can occur either before harvest or after harvest, during storage, on/in the food itself often under warm, damp and humid conditions. Most mycotoxins are chemically stable and survive food processing.

Several hundred different mycotoxins have been identified, but the most commonly observed mycotoxins that present a concern to human health and livestock include aflatoxins, ochratoxin A, patulin, fumonisins, zearalenone and nivalenol/deoxynivalenol.

As seen in Table 2 below, there exists some regulatory guidance as to the allowable levels of mycotoxins in poultry feed. Table 2 presents FDA and European (EU) guidance as to the allowable levels of six mycotoxins in mg/kg: deoxynivalenol (DON); fumonisins (FB); zearalenone (ZEA); aflatoxin (AF); ochratoxin A (OTA); and T-2 toxin (T-2). As shown in the table, no guidance currently exists (NA) for ZEA or T-2 toxin; and the FDA also provides no guidance for OTA.

TABLE 2
Mycotoxin Levels in Poultry Feed Guidance

| | DON | FB | ZEA | AF | OTA | T-2 |
|---|---|---|---|---|---|---|
| FDA Guidance | 5 | 20 | NA | 0.1 | NA | NA |
| EU Guidance | 5 | 20 | NA | 0.02 | 0.1 | NA |

Mycotoxicosis is the disease resulting from exposure to a mycotoxin. The clinical signs, target organs, and outcome depend on the intrinsic toxic features of the mycotoxin and the quantity and length of exposure, as well as the health status of the exposed animal.

The terms “individual,” “subject,” and “animal”, are used interchangeably herein, and refer to animals that are affected by mycotoxicosis. Such animals may be a fish, a bird, a reptile, an amphibian, or a mammal. A bird may be a domesticated or wild fowl, a domestic fowl may be poultry, including a chicken, a duck, or a turkey. A mammal may be a companion animal such as a dog, a cat, a ferret, or an equine; a swine such as a pig, a boar, or a hog; a ruminant such as domestic or wild cattle, including dairy cows and beef cattle, goat, sheep, giraffe, deer, gazelle, or antelope. A reptile may be at least one of a lizard, a turtle, a snake, a chameleon, a Komodo dragon, a crocodile, an iguana, a gecko, or an alligator. An amphibian may be at least one of a salamander, a frog, or a toad.

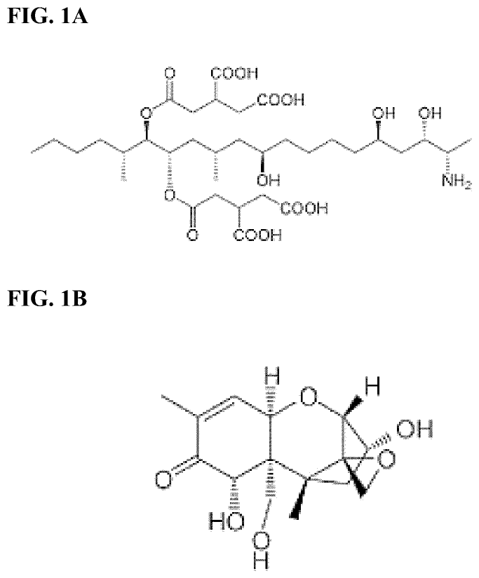

Monogastric livestock, pig and poultry, are particularly vulnerable to mycotoxins because of the high percentage of cereals in their diet, and because they lack a rumen with microbiota able to degrade mycotoxins before their intestinal absorption. From an intestinal pig and poultry health perspective, the most notorious mycotoxins are fumonisins, especially fumonisin B1 (FUM) and trichothecenes, especially deoxynivalenol (DON). The chemical structure of FUM is shown in FIG. 1A, and the chemical structure of DON is shown in FIG. 1B.

Gastropod hemocyanins are massive glycoproteins (4 to 8 MDa) designed by an intricate arrangement of 10 subunits that are self-assembled into hollow cylinders 35 nm in diameter. Hemocyanin is a high molecular weight copper-containing glycoprotein. It reversibly binds oxygen and forms the extra-cellular respiratory protein of mollusks.

As used herein, the term “KLH” is used to refer to the Keyhole limpet hemocyanin of Megathura crenulata. Keyhole limpet hemocyanin (KLH) is filtered from the hemolymph of Megathura crenulata, also called the giant keyhole limpet. It is native to the southern California coast and Mexico. KLH from Megathura crenulata is used as a carrier protein in the production of antibodies.

As used herein, the term “nanoparticle” refers to a particle or a structure in the nanometer (nm) range, typically from about 1 to about 1000 nm in diameter. A nanoparticle is a solid, colloidal particle used to deliver therapeutic agents. Nanoparticles have various properties that facilitate enhanced behavior when compared with larger molecules.

As used herein, the terms “chitosan” and “CS” are used interchangeably and refer to a natural linear polysaccharide composed of randomly distributed β-(1-4)-linked D-glucosamine and N-acetyl-D-glucosamine. Chitosan may be prepared from mushrooms, or by treating shells of sea creatures with an alkaline substance. Chitosan is used in coating medical devices such as stents and catheters, as a facilitator and carrier in targeted drug delivery, in dressings and plasters to accelerate healing, as scaffolding to aid in tissue regeneration, in cosmetics, food preservation and supplements, as excipient in pharmaceuticals, and as antimicrobial agent and growth stimulator in agriculture. In the form of nano-complexes, chitosan has been used for delivering drugs and siRNA. Based on viscosity, chitosan is available as low molecular weight, medium molecular weight, and high molecular weight. Low molecular weight chitosan has an average 50,000 to 190,000 Da molecular weight.

Chitosan nanoparticles can be prepared by any of the various methods known to a person skilled in the art. Chitosan nanoparticle preparation methods include ionic gelation method, emulsification and cross-linking, emulsion droplet coalescence, emulsion solvent diffusion, reverse micellization, ionic gelation, polyelectrolyte complexation, modified ionic gelation with radical polymerization, desolvation (A. Grenha, 2012 “Chitosan nanoparticles: a survey of preparation methods,” J. Drug Targeting 20(4):291-300). Ionic gelation method is very simple and mild and involves reversible physical crosslinking by electrostatic interaction instead of chemical crosslinking, which avoids the possible toxicity of reagents and other undesirable effects. Advantageously, the nanoparticles can be prepared under mild conditions without using harmful solvents, especially organic solvents, which may cause degradation of the antigen as it may be the case if it is a peptide or protein antigen that is unstable and sensitive to other process environments.

As used herein, the term “ionic gelation” refers to complexation between oppositely charged molecules to prepare nanoparticles. In an embodiment of the ionic gelation method chitosan is dissolved in an aqueous medium, which is preferably weak acidic to foster conversion of free amino acid groups (—NH₂) to its positively charged protonized form (—NH₃⁺). Such solution is then combined with an aqueous solution containing negatively charged counterions for example, by addition of an aqueous solution containing chitosan to an aqueous solution containing negatively charged counterions or vice versa. The combination of aqueous solutions is done by dropwise addition of one aqueous solution to the other under constant stirring. Due to the complexation between oppositely charged species, chitosan undergoes ionic gelation and precipitates to form spherical particles. The nanoparticles can be removed by filtration, washed with distilled water and dried.

As used herein, “pharmaceutical composition” refers to a composition that contains a nanoparticle-mycotoxin-hemocyanin complex as disclosed herein, and that is suitable for administration to a subject. The pharmaceutical composition is suitable to prevent, treat, reduce, or ameliorate one or more mycotoxicosis symptoms in the subject. For the purposes of this disclosure, pharmaceutical compositions include vaccines.

As used herein “diluent,” excipient,” “carrier,” and “adjuvant” are used interchangeably, and refer to a diluent, excipient, carrier, or adjuvant which is physiologically acceptable to the subject while retaining the therapeutic properties of the pharmaceutical composition with which it is administered. Physiologically acceptable diluents, excipients, carriers, or adjuvants and their formulations are known to one skilled in the art (see, e.g., U.S. Pat. No. 9,017,691; Chaudhari S. P., et al. 2012, Pharmaceutical Excipients: A Review,” IJAPBC Vol 1(1)). Reed S. G., et al. (2013, “Key Roles of Adjuvants in Modern Medicines,” Nature Medicine 19(12): 1597-1608) review adjuvants used in vaccines.

The terms “inoculated” and “vaccinated” are used interchangeably herein and refer to the act of introducing a nanoparticle-mycotoxin-hemocyanin vaccine disclosed herein to a subject in need thereof.

Any effective route of administration may be utilized to deliver the vaccines comprising nanoparticle-mycotoxin-hemocyanin complexes, such as, for example, orally, nasally, enterally, parenterally, intramuscularly, intravenously, subcutaneously, intradermally, rectally, vaginally, topically, ocularly, pulmonarily, or by contact application. From a practical standpoint, oral, (intra)nasal, parenteral (IM, SubQ, and perhaps intradermal) and ocular may be preferred. In some embodiments, vaccine compositions disclosed herein may be injected (e.g., via intramuscular, intraperitoneal, intradermal and/or subcutaneous routes); or delivered via the mucosa (e.g., to the oral/alimentary, respiratory, and/or genitourinary tracts). In some embodiments, it may be desirable to administer different doses of a vaccine by different routes. The vaccines provided herein can be administered using any appropriate method. Administration can be, for example, topical (e.g. transdermal, ophthalmic or intranasal); pulmonary (e.g., by inhalation or insufflation or powders or aerosols); oral, or parenteral (e.g. by subcutaneous, intrathecal, intraventricular, intramuscular, or intraperitoneal injection, or by intravenous drip). Administration can be rapid (e.g., by injection) or can occur over a period of time (e.g., by slow infusion or administration of slow release formulations). In some embodiments, the mode of administration is intraperitoneal. For application in birds or pigs, the preferred mode of administration is mucosal vaccination, is a suitable approach (nasal, oral, or ocular) to induce a localized immune response in the mucosal tissues. In a commercial poultry operations, the flock sizes contain thousands of birds, and one infected bird is sufficient to increase flock mortality and morbidity. For this reason, it is very important to have efficient methods of mass vaccination to prevent losses. As a vaccination method, the oral delivery route—for example, via feed, water, or oral gavage—has many benefits in the poultry industry. The oral delivery route can: (a) decrease the need for individual administration of vaccines, e.g., intramuscular injections, (b) retain the meat quality of poultry, (c) stimulate mucosal immunity, (d) be rapid, and (e) reduce bird handling, stress, and labor costs.

Vaccine compositions are administered in such amounts and for such time as is necessary to achieve a desired result. As used herein, an “immunogenic” amount of the vaccine composition is an amount which is suitable to elicit an immune response. Thus, the amount effective to treat, attenuate, or prevent disease, as used herein, refers to a nontoxic but sufficient amount of the vaccine composition to treat, attenuate, or prevent disease in any subject. For example, the “therapeutically effective amount” can be an amount to treat, attenuate, or prevent infection (e.g., mycotoxicosis). The exact amount required to achieve an “immunogenic amount” may vary, depending on the particular component (e.g., polysaccharide, conjugate), and from subject to subject, depending on the species, age, and general condition of the subject, the stage of the disease, the particular pharmaceutical formulation, its mode of administration, and the like.

The amount of nanoparticle-mycotoxin-hemocyanin in each vaccine dose is selected to allow the vaccine, when administered as described herein, to induce an appropriate immunoprotective response without significant, adverse side effects. An “immuno-protective” or “protective immune” response as used herein is an immune response sufficient to protect an immunized subject from a toxicity to which a vaccine is directed (e.g., mycotoxin). Optimal amounts of components for a particular vaccine can be ascertained by standard studies involving observation of appropriate immune responses in subjects. Following an initial vaccination, subjects can receive one or several booster immunizations adequately spaced in time. Such amounts may vary depending upon which mycotoxin(s) are in the vaccine, and may be formulated in a unit dosage form for ease of administration and uniformity of dosage. The expression “unit dosage form,” as used herein, refers to a physically discrete unit of vaccine composition appropriate for the subject to be treated.

The term “treating,” as used herein, refers to ameliorating, improving or remedying a disease, disorder, condition or symptom of a disease, disorder, or condition associated with a mycotoxin.

The term “preventing” means to stop or hinder a disease, disorder, condition, or symptom of a disease, disorder, or condition such as mycotoxicosis.

The specific therapeutically effective dose for any particular subject may depend upon a variety of factors including the severity or degree of toxic ingestion; the activity of the specific vaccine or vaccine composition employed; other characteristics of the specific vaccine or vaccine composition employed; the age, body weight, general health, sex of the subject, the diet of the subject, the pharmacokinetic condition of the subject, the time of administration (e.g., with regard to other activities of the subject such as eating, sleeping, receiving other medicines including other vaccine doses, etc.), the route of administration, the rate of excretion of the specific vaccine or vaccine composition employed; vaccines used in combination or coincidental with the vaccine composition employed; and like factors well known in the veterinary arts.

Anti-mycotoxin vaccines for use in accordance with the present disclosure may be formulated according to known techniques. An immunogenic amount of a vaccine product can be formulated together with one or more pharmaceutically acceptable carrier materials (organic, inorganic, liquid, or solid). In general, pharmaceutically acceptable carriers include solvents, dispersion media, and the like, which are compatible with pharmaceutical administration. For example, materials that can serve as pharmaceutically acceptable carriers include, but are not limited to sugars such as lactose, glucose, dextrose, and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; polyols such as glycerol, propylene glycol, and liquid polyethylene glycol; esters such as ethyl oleate and ethyl laurate; agar; buffering agents such as magnesium hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as preservatives, and antioxidants can also be present in the composition, according to the judgment of the formulator (see also Remington's Pharmaceutical Sciences, Fifteenth Edition, E. W. martin (Mack Publishing Co.; Easton, PA, USA 1975).

In some embodiments, at least one booster vaccine is dispensed after the initial administration of the nanoparticle-mycotoxin-hemocyanin vaccine described herein. The booster vaccine may be identical to the vaccine that is initially used to vaccinate the subject. The booster vaccine may be administered about one week after initial vaccination.

One method of detecting whether an adequate immune response has been achieved is to determine seroconversion and antibody titer in the animal after vaccination. The timing of vaccination and the number of boosters, if any, will preferably be determined by a veterinarian based on analysis of all relevant factors, some of which are described above.

Disclosed herein are nanoparticle-mycotoxin-hemocyanin complexes useful in the prevention or amelioration of one or more mycotoxicosis symptoms. In some embodiments of the disclosure, the nanoparticle in the nanoparticle-mycotoxin-hemocyanin complex is chitosan or polyanhydride. In some embodiments the nanoparticle in the nanoparticle-mycotoxin-hemocyanin complex is low molecular weight chitosan. In some embodiments of the disclosure, the hemocyanin is KLH. In some embodiments of the disclosure, the mycotoxin is complexed, encapsulated, partially encapsulated, or associated with one or more nanoparticles. In some embodiments of the disclosure, the mycotoxin is at least one of an aflatoxin; an ochratoxin; a fumonisin (such as fumonosin B1 (FUM)); a zearalenone; or a trichothecene (such as deoxynivalenol (DON) or T-2 mycotoxin). In some embodiments of the disclosure, the nanoparticle-mycotoxin-hemocyanin complex has two mycotoxins.

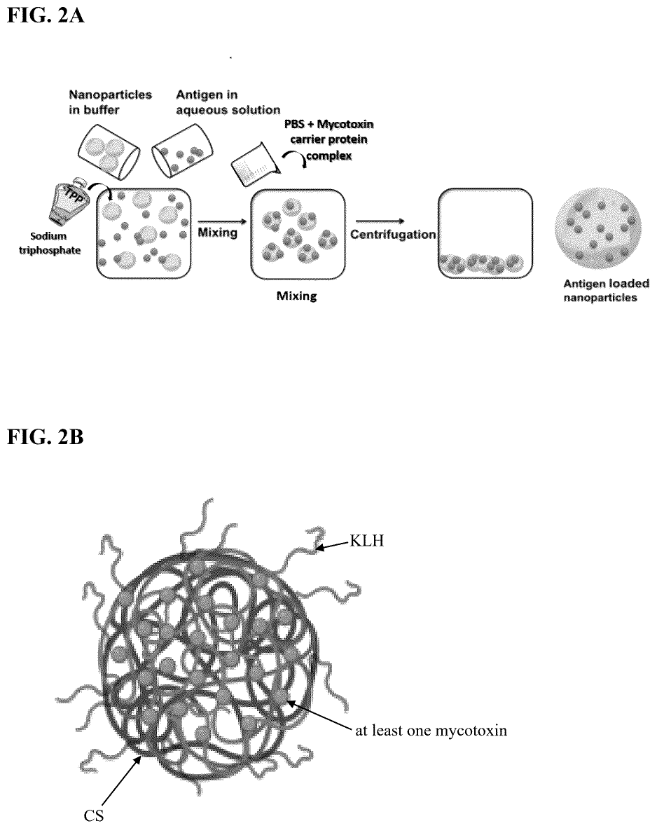

Described herein is the preparation and use of an immunogenic composition comprising a nanoparticle-mycotoxin-hemocyanin complex. The schematic shown in FIG. 2A, depicts the process used in the preparation of nanoparticle-mycotoxin-hemocyanin complexes. A diagrammatic representation of a nanoparticle-mycotoxin-hemocyanin complex is shown in FIG. 2B.

As disclosed in Example 1, a nanoparticle-based mycotoxin immunogenic complex was prepared using an ionic gelation method. Briefly, FUM-KLH and DON-KLH were added to low molecular weight chitosan pH4.3 dissolved in glacial acetic acid, followed by the addition of sodium tripolyphosphate (TPP), the complex were separated by centrifugation, resuspended in buffer, lyophilized, and stored at −20° C. until used. To prepare the vaccine, 100 ng CS-FUM-DON-KLH in 1 mL PBS (pH 7.2) was mixed with 1:1 with complete Freund's adjuvant for a primary vaccine, or with incomplete Freund's adjuvant for a booster vaccine.

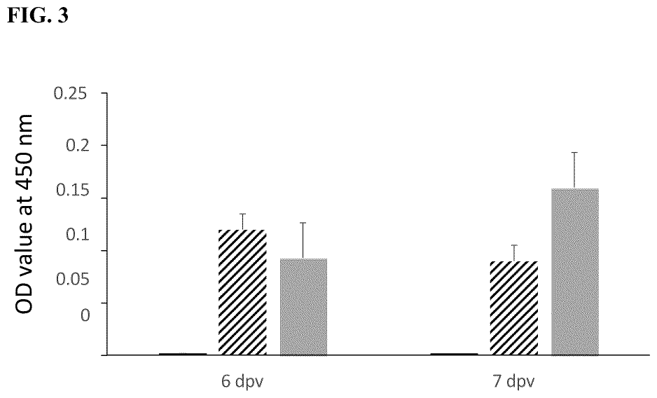

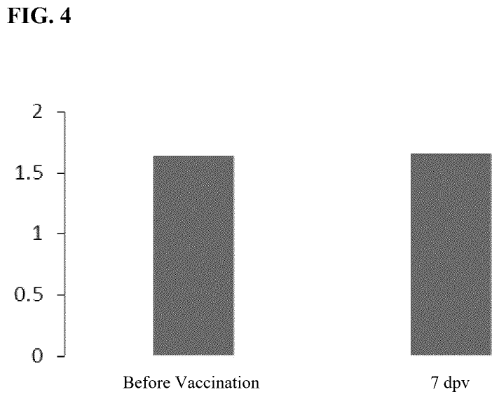

Example 2 presents layer bird vaccine trial results. This Example shows that anti-FUM IgY and anti-DON IgY were detected at 6 and 7 days post-vaccination in layer birds fed regular diet (see FIG. 3). Furthermore, the nanoparticle vaccine did not cause adverse effects on the treated chickens. As seen in FIG. 4, there was no visible weight loss, a 0% mortality, no detectable swelling at the injection site, and egg production was similar to the one prior to vaccination. This Example showed that a single dose of a CS-FUM-KLH and CS-DON-KLH vaccine successfully induced anti-FUM and anti-DON antibodies, and that a booster dose increased these antibodies in vivo.

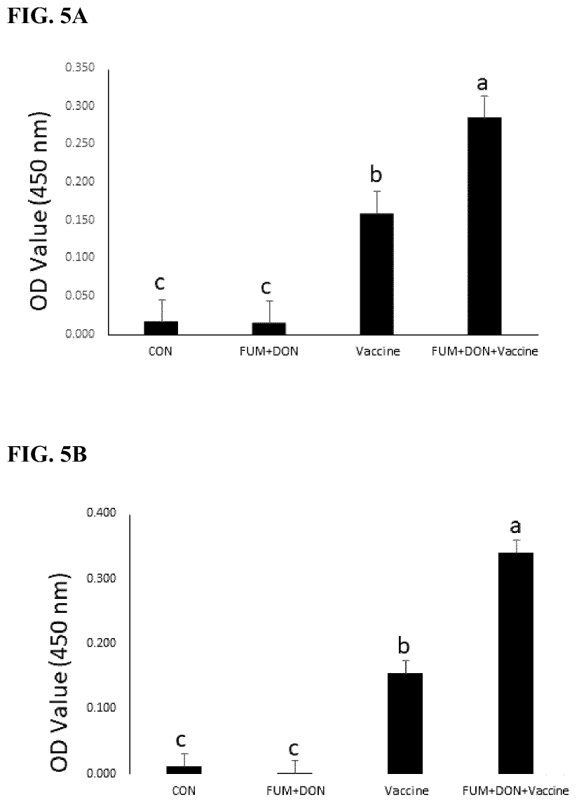

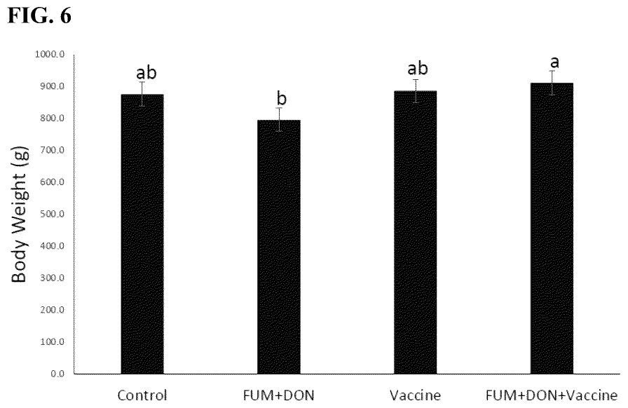

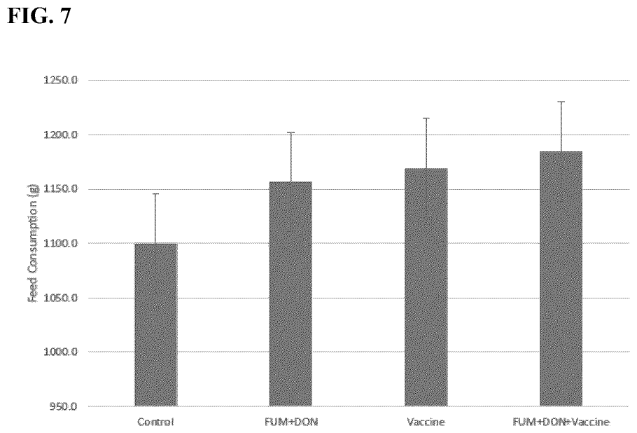

Example 3 presents broiler bird vaccine trial results. This Example shows that no anti-FUM IgY or anti-DON IgY was detectable in non-vaccinated animals fed FUM and DON, while they were detected in vaccinated animals fed both, regular diet and a diet supplemented with FUM and DON (FIG. 5A and FIG. 5B). Twenty one days post vaccination, the weight of non-vaccinated birds fed regular diet was the same as that of vaccinated birds fed regular diet, non-vaccinated birds fed diet supplemented with FUM and DON had lost an average of 80.2 grams per bird, and vaccinated birds that were fed a diet supplemented with FUM and DON gained more weight than vaccinated or non-vaccinated birds fed regular diet (FIG. 6). The presence of FUM and DON in the feed appeared to improve appetite, as was vaccination with CS-FUM-DON-KLH. As seen in FIG. 7, the food consumption of vaccinated birds feed a diet supplemented with FUM+DON was highest, with vaccinated birds fed a regular diet next, non-vaccinated birds feed a diet supplemented with FUM and DON next, and non-vaccinated birds fed a non-supplemented diet eating the least.

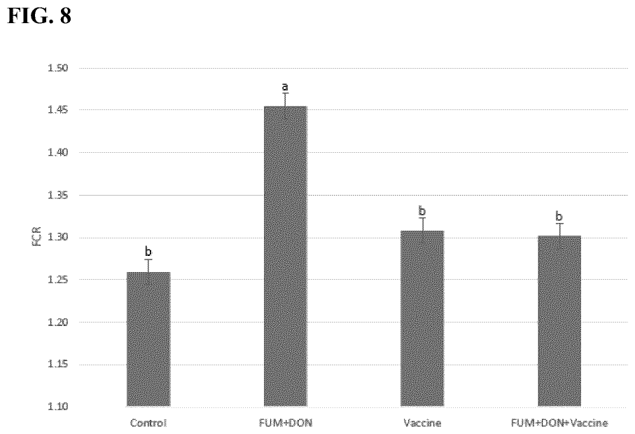

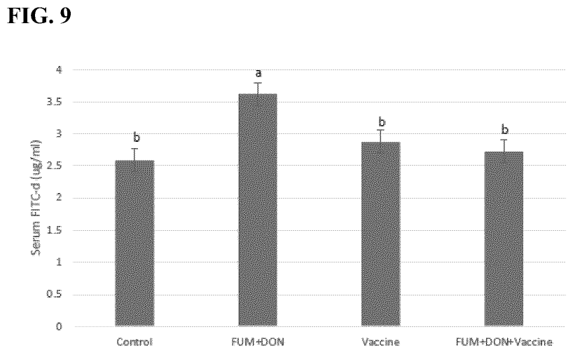

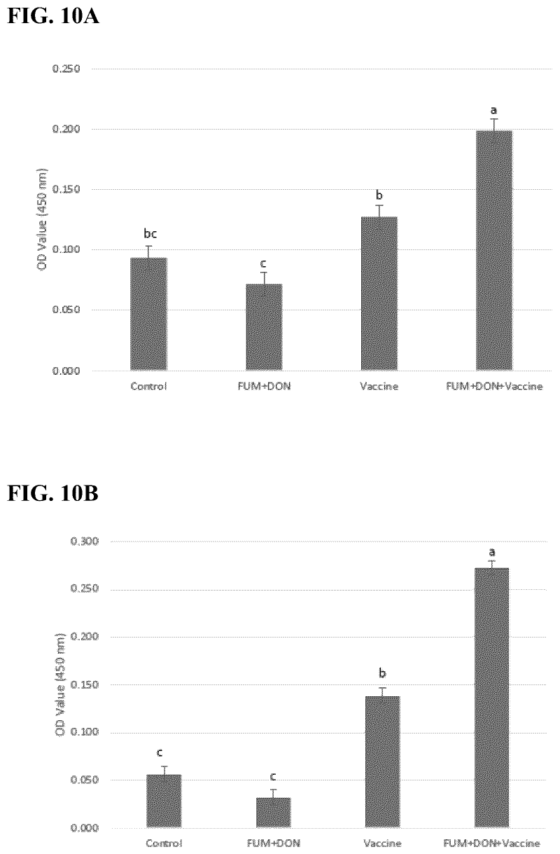

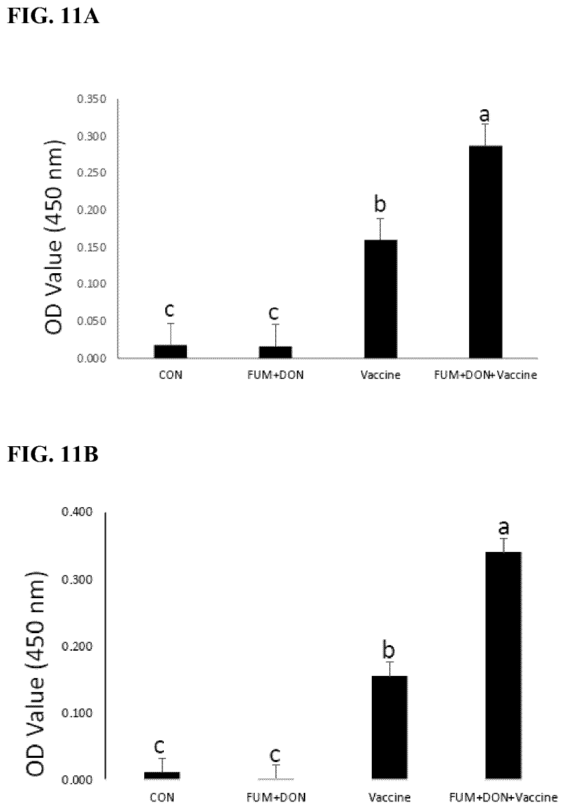

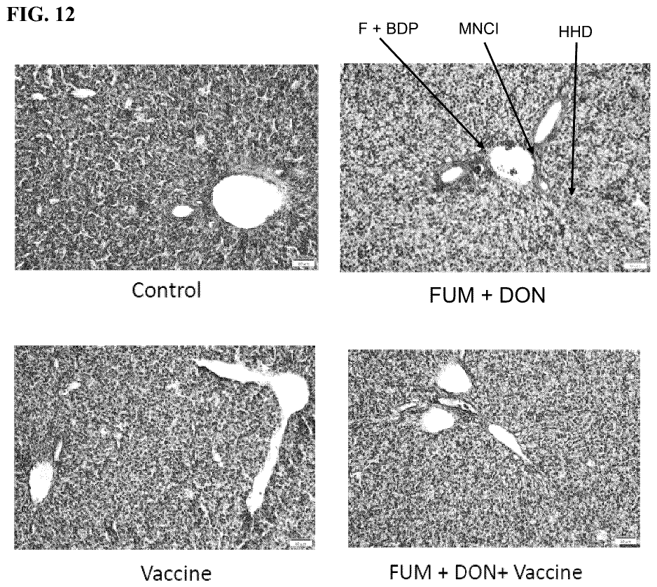

As seen on FIG. 8, mycotoxins worsened feed conversion rate (FCR) by body weight by 19 points. This effect was reversed in birds vaccinated with CS-FUM-DON-KLH fed a regular diet or fed a diet supplemented with FUM and DON. Mycotoxins increased serum FITC-d by body weight by 1.03 g/mL as measured at day 21. FIG. 9 shows that the CS-FUM-DON-KLH vaccine reversed the mycotoxin-induced loss in gut integrity in birds fed with regular diet, or fed regular diet supplemented with FUM and DON. As seen in FIG. 10A and FIG. 10B, DON and FUM, being chemicals did not induce anti-DON or anti-FUM antibodies in bile obtained at day 21 from birds exposed to DON and FUM through feed. Anti-DON and anti-FUM antibodies were detected in birds fed a regular diet and vaccinated with CS-DON-KLH or CS-FUM-KLH. Exposure to DON and FUM through feed further increased the anti-DON and anti-FUM antibodies in vaccinated birds. Similar results were attained for serum obtained at day 21 (see FIG. 11A and FIG. 11B). Vaccination reversed decrease in villi height and crypt depth at the jejunal (intestinal) villi caused by FUM and DON in feed. As seen in FIG. 12, the damage caused by diet supplemented with FUM and DON was reversed upon vaccination with CS-FUM-KLH and CS-DON-KLH.

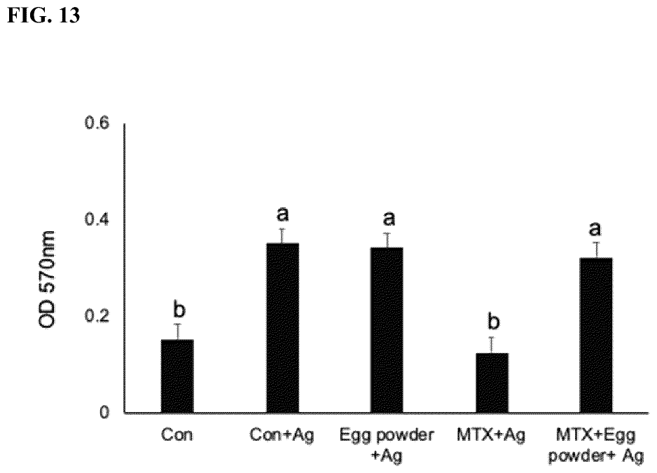

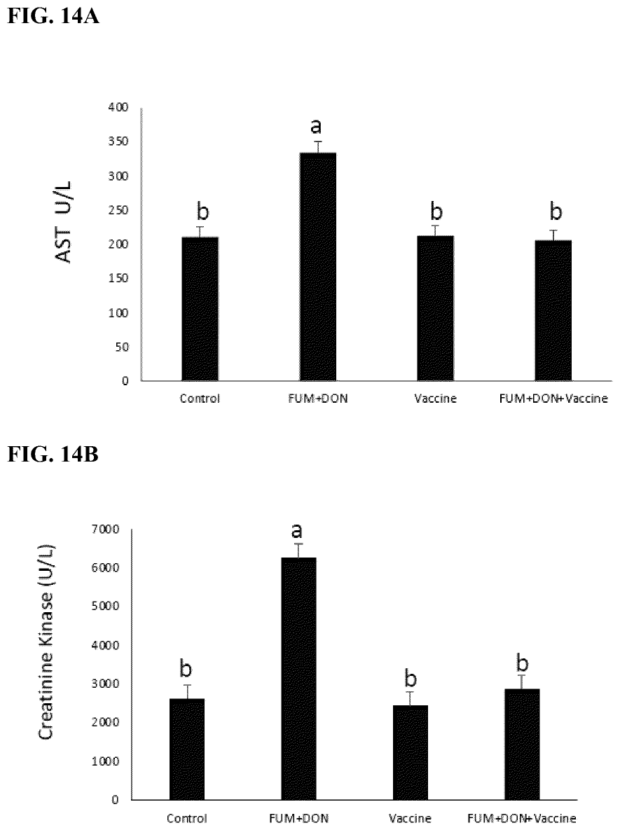

The recall assay is an assay used for quantifying antigen-specific memory T-cells. The data in FIG. 13 shows that the groups vaccinated with the nanoparticle vaccine had slightly higher FUM and DON-specific memory lymphocytes, indicating a successful vaccine. As seen in FIG. 14A and FIG. 14B, the levels of serum aspartate aminotransferase and creatine kinase increased in birds fed a diet supplemented with FUM and DON, while they were very similar in control and vaccinated birds. The presence of FUM and DON in the feed did not appear to make a difference. In summary, the nanoparticle vaccine reversed FUM and DON-induced loss in body weight, increase in FCR, disruption of gut integrity, decrease in jejunal villi height and crypt depth, liver damage, and increase in serum aspartate aminotransferase and creating kinase.

Antibodies against Salmonella enteritidis, S. typhimurium, E. coli, and several gut pathogens are produced in egg yolks. It is shown here that feed supplemented with powder obtained from eggs expressing anti-mycotoxin antibodies prevents or ameliorates symptoms associated with mycotoxicosis in an animal in need thereof animals. Described herein is the characterization and use of a hyperimmunized egg product useful in the prevention or amelioration of mycotoxicosis.

Egg-producing animals produce antibodies in blood and eggs that are specific to particular immunogens. For example, various genera of the class Aves, such as chickens (Gallus domesticus), turkeys, and ducks produce antibodies against antigens associated with avian diseases. Polson A., et al. (1980, “Antibodies to Proteins from Yolk of Immunized Hens,” Immunol. Commun. 9(5): 495-514) immunized hens against several proteins and natural mixtures of proteins, and detected IgY antibodies in the yolks of the eggs. Fertel R., et al. (1981, “Formation of antibodies to prostaglandins in the yolk of chicken eggs,” Biochem. Biophys. Res. Comm. 102:1028-1033) immunized hens against prostaglandins and detected antibodies in the egg yolk. Jensenius J. R. et al. (1981, “Eggs: Conveniently packaged antibodies. Methods for purification of yolk IgG,” J. Immunol. Meth. 46:63-68) provide a method of isolating egg yolk IgG for use in immunodiagnostics. Polson A. et al. (1980, “Isolation of Viral IgY Antibodies from Yolks of Immunized Hens,” Immun. Commun. 9: 475-493) describe antibodies isolated from the yolk of hens that were immunized with a variety of plant viruses.

U.S. Pat. No. 4,748,018 discloses a method of passively immunizing a mammal an animal against a condition caused by an antigen. The method comprises administering to the mammal immunizing amounts of an antibody obtained from a domesticated fowl which has been immunized against the antigen; the mammal being tolerant to the antibody by virtue of having a history of consumption of antibody containing material derived from the egg of a fowl.

U.S. Pat. No. 5,772,999 discloses a method of preventing, countering or reducing chronic gastrointestinal disorders or Non-Steroidal Anti-Inflammatory Drug-induced (NSAID-induced) gastrointestinal damage in a subject by administering to the subject hyperimmunized egg, hyperimmunized milk, or fractions thereof.

U.S. Pat. Nos. 11,230,590 and 10,450,364 relate to a hyperimmunized egg produced by an animal that has been hyperimmunized with at least one isolated antigen selected from Clostridium perfringens α-toxin, elongation factor T, necrotic enteritis B-like toxin, Pyruvate: Ferredoxin oxidoreductase, and Eimeria tenella elongation factor 1-alpha; and a method for preventing or treating necrotic enteritis by administering a hyperimmunized egg product obtained from such an egg to an animal.

The egg yolk antibody platform is economical, ethical, and does not have animal welfare issues. There are a number of advantages to using chickens for the production of antibodies. Chickens present a much more economical source of large quantities of specific antibodies. A 1994 study estimated that the productivity of antibodies from yolk was nearly 18 times greater than that from rabbits, based on the weight of antibody produced per animal. IgY production is also less invasive, requiring only the daily collection of eggs compared to blood collection in mammals. In contrast to mammalian serum, egg yolk contains only a single class of antibody (IgY), which can easily be isolated from the yolk by precipitation techniques. Although the amount of IgY deposited into the yolk varies depending on several factors, including the age, breed of chicken, and antigen used, IgY yields have been reported to range from 60 to 150 mg IgY per egg. IgY has been used as a feed additive for livestock to both target specific pathogens and improve growth and feed efficiency. The vaccination of hens and automated collection and processing of eggs is already carried out on an industrial scale, making the large-scale production of IgY technically feasible.

Chicken egg yolk antibodies are commercially produced against Salmonella spp., E. coli, bovine rotavirus, infectious bursal disease virus, porcine epidemic diarrhea virus, Elmeria spp., canine parvovirus, among others.

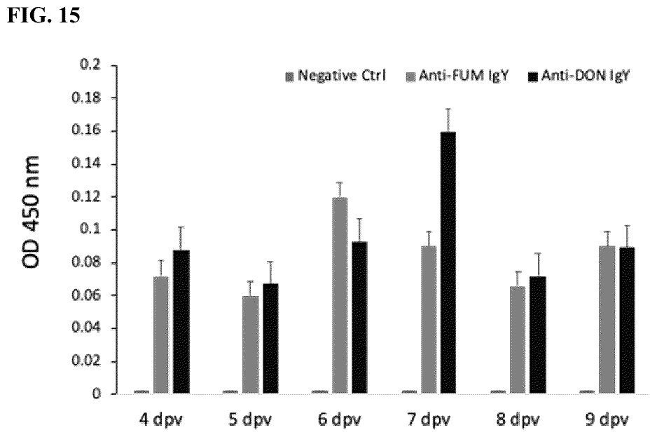

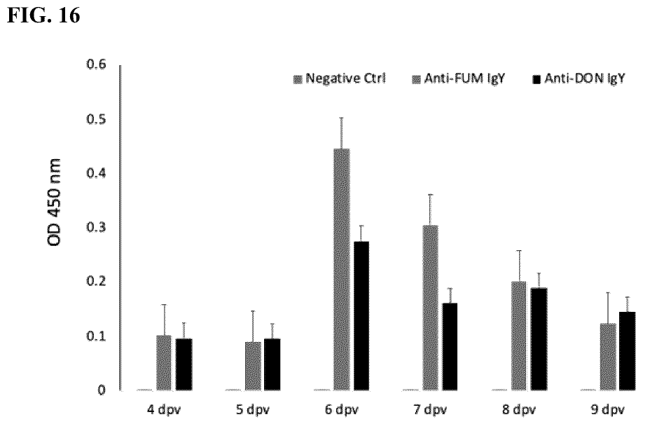

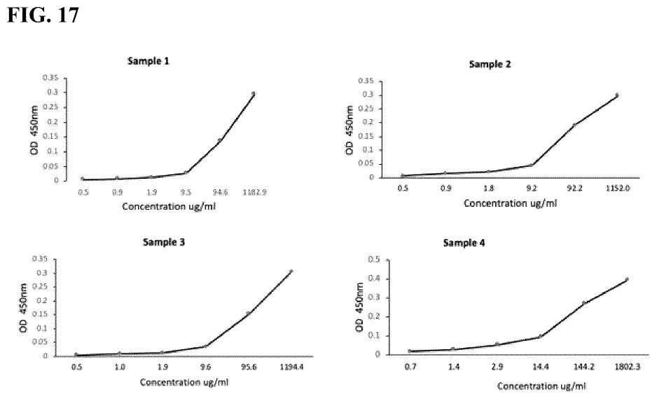

Example 4 shows that layer birds vaccinated with anti-mycotoxin vaccine transfer antibodies to egg yolk. As seen in FIG. 15, no IgY was detected in serum from chickens not vaccinated with CS-FUM-KLH and CS-DON-KLH, while anti-FUM IgY, and anti-DON IgY were detected in serum from vaccinated chickens at all timepoints measured, with an anti-FUM IgY peak around 6 dpv, and an anti-DON IgY peak around 7 dpv. Detection of IgY in egg yolks presented with a similar pattern. As seen in FIG. 16, no IgY was detected in eggs from chickens not vaccinated with CS-FUM-KLH and CS-DON-KLH, while anti-FUM IgY and anti-DON IgY were detected in eggs from vaccinated chickens at all timepoints measured, with a peak around 6 dpv. As seen in FIG. 17, FUM was detected at an egg yolk concentration of about 9 μg/mL to about 10 μg/mL.

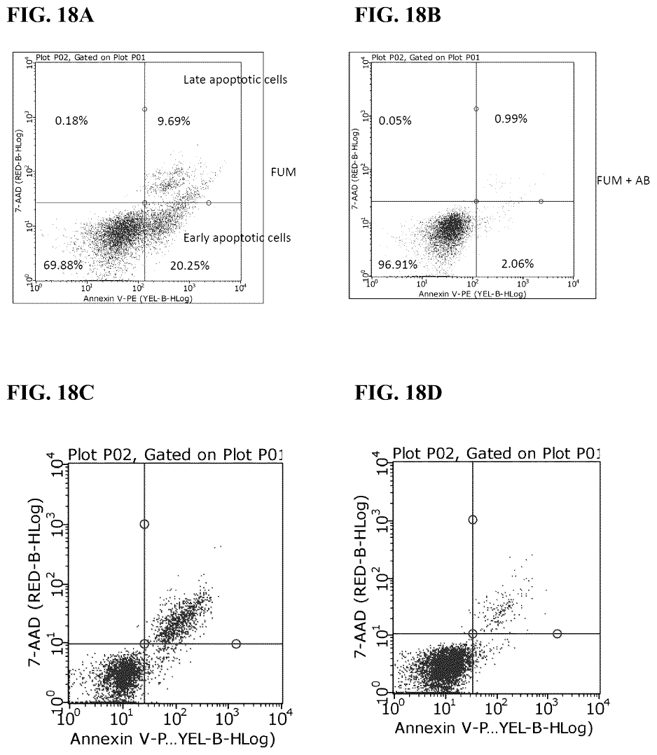

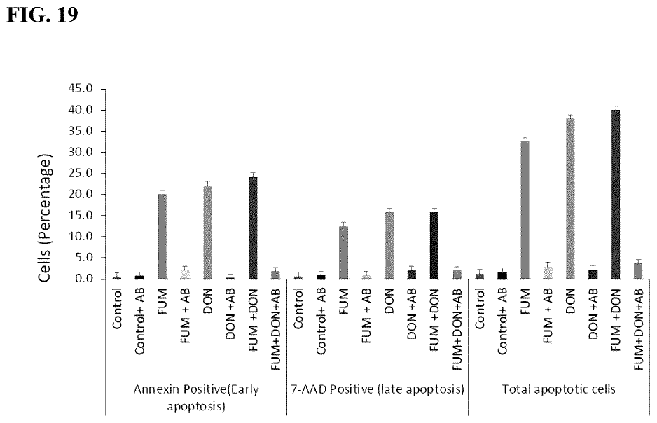

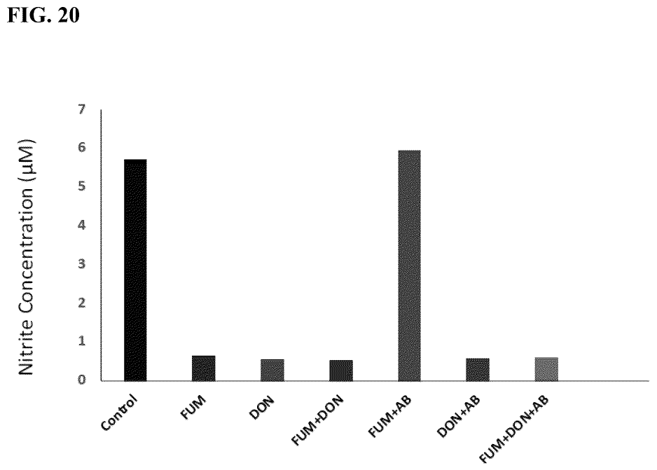

Example 5 shows the in vitro effect of FUM, DON, and/or egg yolk powder from CS-FUM-KLH and CS-DON-KLH-vaccinated chickens were tested on the chicken macrophage cell line, HD11 cells. The data in FIG. 18A shows that FUM induces apoptosis in 30% of HD11 cells, and FIG. 18B shows that addition of egg yolk powder obtained from eggs of anti-mycotoxin-vaccinated chickens reversed the FUM effect on the HD11 cells. Similarly, FIG. 18C shows that DON induces apoptosis in 50% of HD11 cells, and FIG. 18D shows that addition of egg yolk powder obtained from eggs of anti-mycotoxin-vaccinated chickens reversed the DON effect on the HD11 cells. The data in FIG. 19 shows that addition of FUM, DON, or FUM and DON induce early and late HD11 cell apoptosis, but these effects are reversed when egg yolk powder from eggs laid by anti-FUM/DON vaccinated chickens. As seen on FIG. 20, FUM decreases NO production by macrophage cell lines as expected, but addition of egg yolk powder from eggs laid by anti-FUM/DON vaccinated chickens reverses this effect.

Example 6 shows the bile, gut integrity, jejunal histology, jejunal tight junction protein analysis, liver histology, blood chemistry, recall assay, and serum enzyme activity of broiler birds were determined on day 21. Broiler birds were fed egg yolk from layer chickens vaccinated with CS-FUM-KLH and CS-DON-KLH on day 0 followed by a booster on day 7. Production performance data was gathered on days 7, 14, and 21, and serum was collected on days 14 and 21.

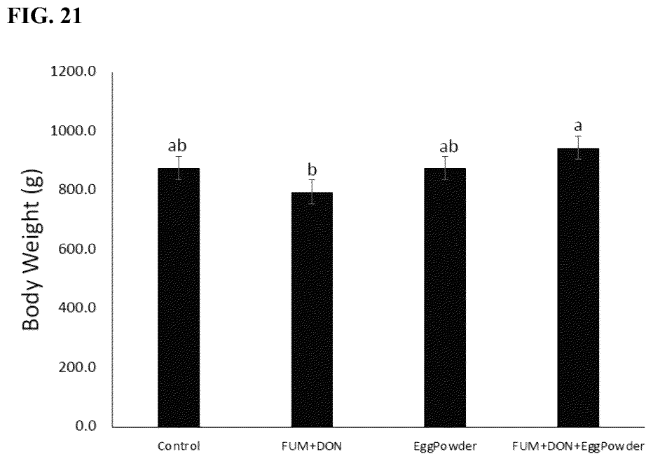

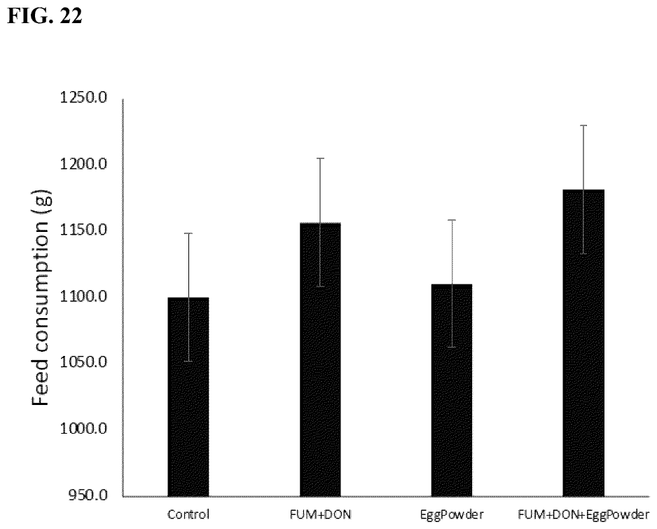

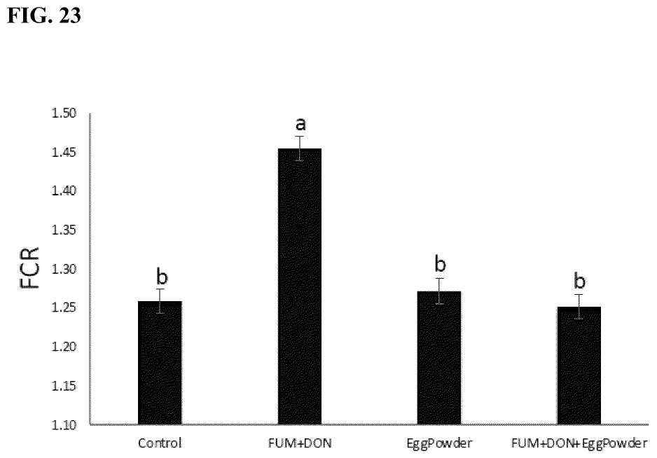

As seen in FIG. 21, mycotoxins decreased body weight at d21 by 80.2 g per bird. Addition of egg yolk powder obtained from eggs of CS-FUM-DON-KLH-vaccinated chickens reversed this effect (student t test; P=0.10). FIG. 22 shows that addition of FUM and DON numerically increased feed consumption at day 21, and this effect was not reversed when the chickens were fed egg yolk powder obtained from eggs of CS-FUM-DON-KLH-vaccinated chickens. The data in FIG. 23 shows that mycotoxins worsened FCR by body weight by 19 points, but this effect was reversed when the chickens were fed a diet supplemented with egg yolk powder obtained from eggs of CS-FUM-DON-KLH-vaccinated chickens.

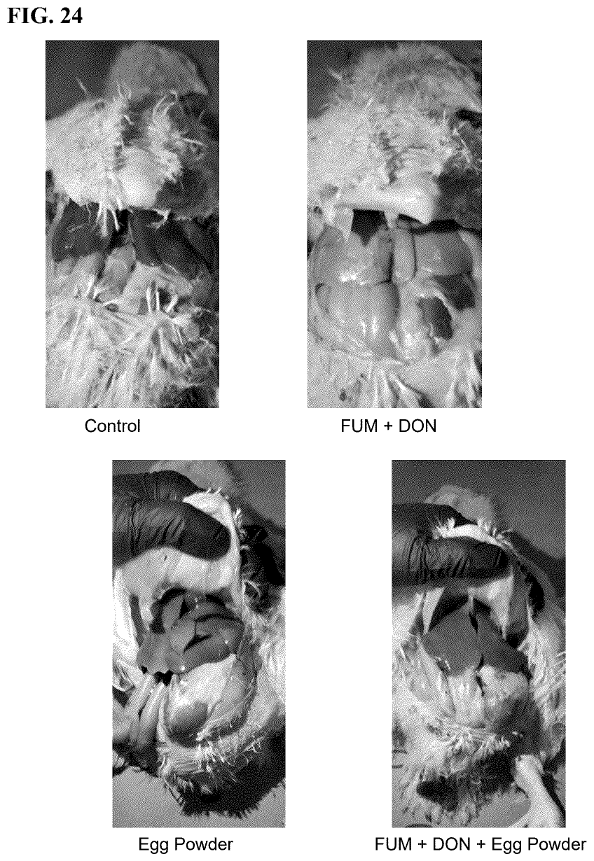

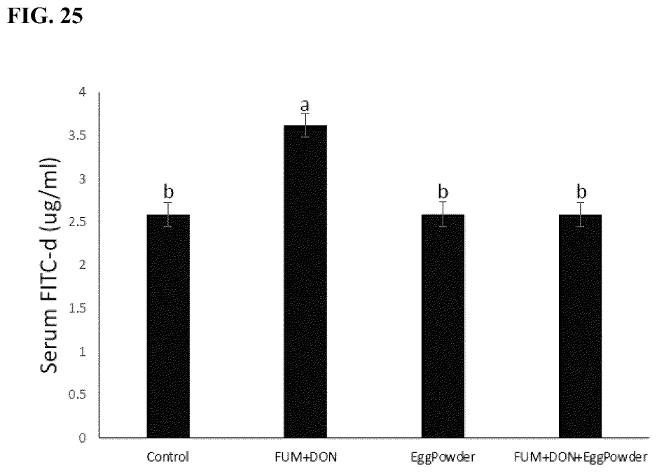

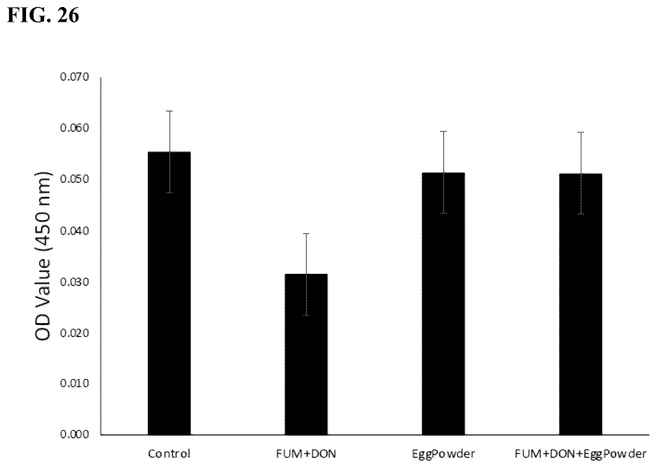

As seen in FIG. 24, the liver from chickens fed diet supplemented with mycotoxins appeared lighter than those from chickens fed regular diet, diet supplemented with egg yolk powder obtained from eggs of CS-FUM-DON-KLH-vaccinated chickens, or fed diet supplemented with FUM, DON, and egg yolk powder obtained from eggs of CS-FUM-DON-KLH-vaccinated chickens. FIG. 25 shows that addition of mycotoxins to the feed increased serum FITC-d by body weight by 1.03 μg/mL, while addition of egg yolk powder obtained from eggs of CS-FUM-DON-KLH-vaccinated chickens to the feed, either alone or in the presence of mycotoxins, reversed this effect. Because egg yolk powder acts by neutralizing the mycotoxins in the gut, as seen in FIG. 26, as expected, adding egg yolk powder obtained from eggs of CS-FUM-DON-KLH-vaccinated chickens to the feed did not increase the anti-FUM bile IgA.

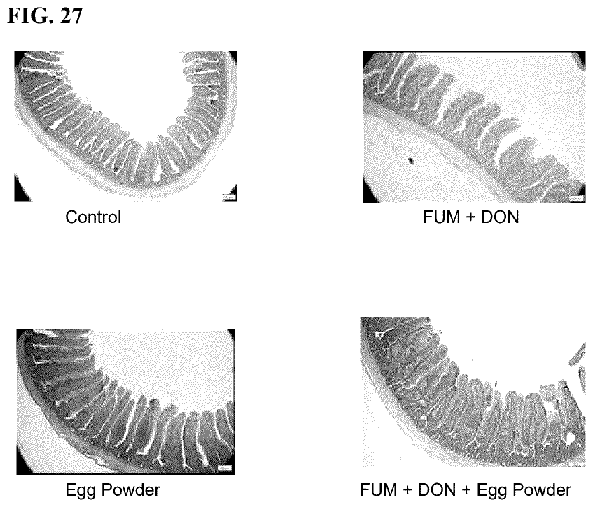

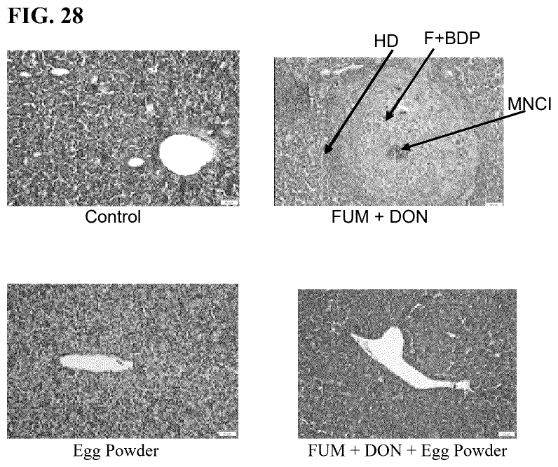

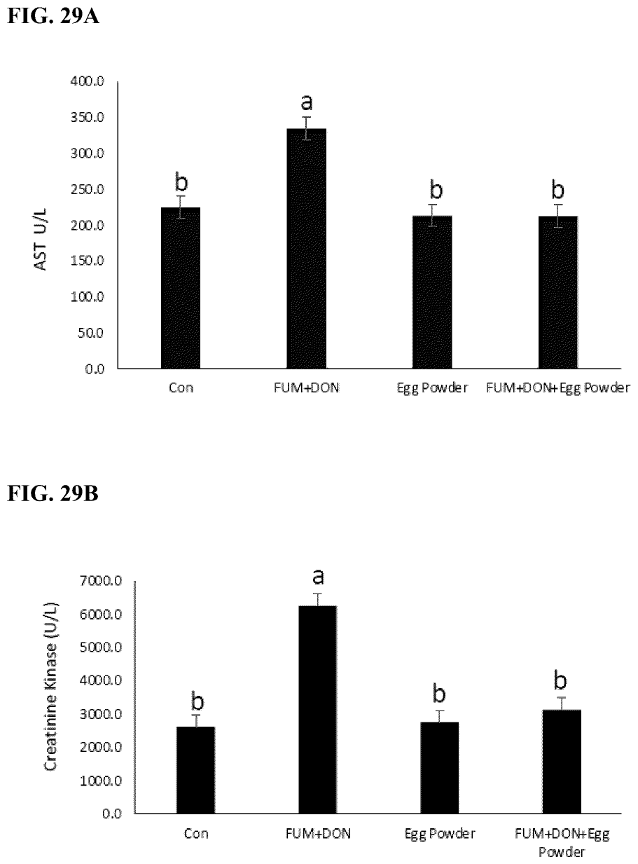

As seen in the se images presented in FIG. 27, addition of FUM and DON to the chicken feed induced decrease in villi height and crypt depth at the jejunal (intestinal) villi. This effect was reversed by the addition to the feed of egg yolk powder obtained from eggs of CS-FUM-DON-KLH-vaccinated chickens. FIG. 28 depicts images of Liver sections from layer birds fed regular diet (Control), fed a diet supplemented with FUM and DON (FUM+DON), fed a diet supplemented with egg yolk powder containing anti-FUM and anti-DON IgY (Egg Powder), or fed a diet supplemented with FUM and DON and egg yolk powder containing anti-FUM and anti-DON IgY (FUM+DON+Egg Powder). The mononuclear cell infiltration (MNCI), fibrosis and bile duct proliferation (F+BDP), and the hydropic degeneration (HD) are indicated by arrows in the image of the liver section from chickens fed a diet supplemented with FUM and DON. These damages were reversed in chickens fed diets supplemented with egg yolk powder containing anti-FUM and anti-DON IgY Graphs depicting the amounts of serum aspartate aminotransferase and creatine kinase detected in layer chickens at day 21 after different treatments are shown in FIG. 29A and FIG. 29B. These graphs show that addition of only FUM and DON to the feed increases the amounts of aspartate aminotransferase and creatine kinase, while addition of egg yolk powder containing anti-FUM and anti-DON IgY reverses these effects.

Mention of trade names or commercial products in this disclosure is solely for the purpose of providing specific information and does not imply recommendation or endorsement by the U.S. Department of Agriculture.

While this disclosure may be embodied in many different forms, there are described in detail herein specific preferred embodiments. The embodiments disclosed herein are an exemplification of the principles of the disclosure, and are not intended to limit the disclosure to the particular embodiments illustrated. All patents, patent applications, scientific papers, and any other referenced materials mentioned herein are incorporated by reference to the same extent as if each individual patent, patent application, scientific paper, or any other referenced material was specifically and individually indicated to be incorporated by reference. Furthermore, the disclosure encompasses any possible combination of all or some of the various embodiments and characteristics described herein and/or incorporated herein. In addition, the disclosure encompasses any possible combination that also specifically excludes any one or some of the various embodiments and characteristics described herein and/or incorporated herein.

Thus, in view of the above, there is described (in part) the following:

An immunogenic composition comprising at least one mycotoxin bound to a carrier protein and entrapped within a nanoparticle.

The above immunogenic composition, wherein the at least one mycotoxin is at least one of an aflatoxin, an ochratoxin, a fumonisin, a zearalenone, or a trichothecene.

The above immunogenic composition, wherein the at least one mycotoxin is a fumonosin, wherein said fumonosin is fumonosin B (FUM).

The above immunogenic composition, wherein the at least one mycotoxin is a trichothecene, wherein said trichothecene is deoxynivalenol (DON) or T-2 mycotoxin.

The above immunogenic composition, wherein the at least one mycotoxin is FUM and DON.

The above immunogenic composition, wherein the nanoparticle comprises chitosan.

The above immunogenic composition, wherein the carrier protein is Keyhole limpet hemocyanin (KLH).

The above immunogenic composition, optionally comprising an adjuvant or carrier.

A method of treating an animal in need thereof to prevent or ameliorate one or more symptoms of mycotoxicosis, the method comprising administering to the animal at least one effective dose of a composition comprising at least one mycotoxin bound to a carrier protein and entrapped within a nanoparticle.

The above method, wherein the method protects the animal from one or more clinical signs of mycotoxin-induced mycotoxicosis.

The above method, wherein the animal is a fish, a bird, a reptile, an amphibian, or a mammal.

The above method, wherein the animal is a swine or a fowl.

The above method, wherein the at least one mycotoxin is an aflatoxin, an ochratoxin, a fumonisin, a zearalenone, or a trichothecene.

The above method, wherein the carrier protein is KLH.

The above method, wherein the nanoparticle comprises a chitosan.

The above method, wherein the composition is administered to the animal at least twice.

The above method, wherein the composition is administered to the animal intramuscularly, orally, and/or intradermally.

A method for preparing a composition comprising at least one mycotoxin bound to a carrier protein and entrapped within a nanoparticle, the method comprising: linking at least one mycotoxin to a carrier protein to prepare a mycotoxin-carrier protein complex; adding the mycotoxin-carrier protein complex drop by drop into a nanoparticle solution and stirring for a determined amount of time; adding sodium tripolyphosphate as a linking agent; and collecting the nanoparticle-mycotoxin-carrier protein complex, wherein the at least one mycotoxin is bound to the carrier protein and entrapped within the nanoparticle.

The above method, wherein the carrier protein is a hemocyanin.

The above method, wherein the hemocyanin is Keyhole limpet hemocyanin (KLH) and the nanoparticle comprises a chitosan.

A composition comprising egg or a part thereof enriched in at least one anti-mycotoxin antibody. An egg enriched in at least one anti-mycotoxin antibody comprises a larger amount of anti-mycotoxin IgY. An egg enriched in at least one anti-mycotoxin antibody is a hyperimmune egg.

The above composition, wherein the egg or part thereof enriched in at least one anti-mycotoxin antibody is from an egg-producing animal vaccinated with an immunogenic composition comprising a mycotoxin. The egg or part thereof enriched in at least one anti-mycotoxin antibody is a hyperimmune egg, and comprises a larger amount of anti-mycotoxin IgY than a non-hyperimmune egg.

The above composition, wherein the at least one anti-mycotoxin antibody neutralizes aflatoxin, ochratoxin, fumonisin, zearalenone, or trichothecene. The above composition, wherein the at least one anti-mycotoxin antibody neutralizes at least FUM or DON.

A feed supplement comprising the above composition.

The above feed supplement for administration to a fish, a bird, an amphibian, a reptile, or a mammal.

The above feed supplement, wherein the bird is a domesticated or wild fowl bird.

The above feed supplement, wherein the mammal is a wild or domesticated mammal.

The above feed supplement, wherein the wild or domesticated mammal is a swine.

A feed comprising feed material and the above feed supplement.

The above feed, wherein the feed comprises at least about 0.0001%, at least about 0.0005%; at least about 0.0010%; at least about 0.0020%; at least about 0.0025%; at least about 0.0050%; at least about 0.0100%; at least about 0.020%; at least about 0.100% at least about 0.200%; at least about 0.250%; at least about 0.500%, at least about 0.750%, at least about 1.00%, at least about 1.50%, at least about 1.75%, at least about 2.00%, at least about 3.00%, at least about 4.00%, at least about 5.00%, at least about 6.00%, at least about 7.00%, at least about 8.00%, at least about 9.00%, or at least about 10.00%, by weight of the feed supplement.

The above feed, further comprising at least one additive.

The above feed, wherein the feed is in the form of powder, granules, pellets, tablets, capsules, gel, paste, emulsion, liquid, chew bars, chew bones, chew rolls, or treats.

A method for preparing a composition comprising at least one anti-mycotoxin antibody, the method comprising: collecting eggs laid by chickens vaccinated with at least one anti-mycotoxin vaccine, or collecting egg yolks or egg whites from such eggs; and drying the collected eggs, egg yolks, or egg whites.

The above method, wherein the anti-mycotoxin vaccine comprises at least one mycotoxin bound to a carrier protein and entrapped within a nanoparticle.

The above method, wherein the at least one mycotoxin is an aflatoxin, an ochratoxin, a fumonisin, a zearalenone, or a trichothecene.

The above method, wherein the carrier protein is hemocyanin.

The above method, wherein the carrier protein is Keyhole limpet hemocyanin.

The above method, wherein the nanoparticle comprises chitosan.

A method of preventing or ameliorating one or more symptoms of mycotoxicosis in an animal in need thereof, the method comprising administering to the animal at least one dose of a composition comprising an egg or part thereof enriched in at least one anti-mycotoxin antibody, wherein feeding the composition to the animal protects the animal from one or more clinical signs of mycotoxin-induced mycotoxicosis.

The above method, wherein the composition is provided to the animal in feed or drinking water.

Embodiments of the present disclosure are shown and described herein. It will be obvious to those skilled in the art that such embodiments are provided by way of example only. Numerous variations, changes, and substitutions will occur to those skilled in the art without departing from the disclosure. Various alternatives to the embodiments described herein may be employed in practicing the disclosure. It is intended that the included claims define the scope of the disclosure and that methods and structures within the scope of these claims and their equivalents are covered thereby.

### EXAMPLES

Having now generally described this invention, the same will be better understood by reference to certain specific examples, which are included herein only to further illustrate the invention and are not intended to limit the scope of the invention as defined by the claims.

### Example 1

### Nanoparticle Vaccine Preparation

A nanoparticle vaccine that recognizes fumonisin (FUM) and deoxynavilenol (DON) was prepared.

FUM and DON haptens were linked to Keyhole limpet hemocyanin (KLH) to convert them into immunogens. Briefly, 10 mg of KLH was dissolved in 1 mL of PBS (pH 7.2). 70 μL of 3 mg of M-maleimidobenzoic acid N-hydroxy succinimide ester (MBS) in 200 μL of dimethyl formamide (DMF) solution was added drop by drop to 500 μL of KLH and incubated at room temperature for 1 hour. This KLH-MBS solution was passed through a PD-10 desalting column and washed using PBS with pH 6.5. The column was centrifuged at 350×g for 2 minutes to collect the KLH-MBS solution. Ten mg of fumonisin B1 (FUM) and 10 mg of deoxynivalenol was dissolved in 200 μL of DMF. One hundred μL of FUM and 100 μL of DON was mixed with 800 μL of KLH-MBS solution and vortexed for 30 seconds. The pH was adjusted to 7.2 and left in a shaker at 4° C. overnight. Three mL of 0.1 M ammonium bicarbonate was added and lyophilized. The lyophilized powder was dissolved in sucrose solution, and stored at −20° C. until used.

Chitosan nanoparticle-based vaccines comprising FUM and/or DON (CS-FUM-KLH and CS-DON-KLH, or CS-FUM-DON-KLH) were prepared using an ionic gelation method. Briefly, 100 mg low molecular weight chitosan (Sigma-Aldrich, St Louis, MO, USA) was dissolved in 4% glacial acetic acid with intermittent sonication and the pH was adjusted to 4.3. The solution was stirred until the solution was transparent. Ten mg/mL (1% W/V) chitosan working stock was prepared in molecular biology grade water. This chitosan solution was filtered by passing it through 0.22 μm filter. One mL of FUM-KLH and/or DON-KLH solution was added drop by drop into the chitosan solution and stirred for 1 hour at room temperature. A 1% (W/V) sodium tripolyphosphate (TPP) solution was used as a cross-linking agent by adding 1 mL TPP to the CS-FUM-KLH and/or CS-DON-KLH solution and continued to stir for 30 minutes. The resulting chitosan nanoparticles (CS-FUM-DON-KLH, CS-FUM-KLH, or CS-DON-KLH) were purified by centrifugation at 10,500×g for 30 minutes. After removal of the supernatant, the pellets were resuspended in PBS (pH 7.4) containing 5% sucrose, lyophilized, and stored at −20° C. until used.

FIG. 2A shows a schematic diagram for the preparation of a CS-FUM-DON-KLH, CS-FUM-KLH, or CS-DON-KLH, and FIG. 2B shows a schematic diagram of an antigen-loaded CS nanoparticle.

To prepare the vaccine, 4000 ng of a CS-FUM-DON-KLH in 1 mL PBS (pH 7.2) was mixed with 1:1 with complete Freund's adjuvant for a primary vaccine, or with incomplete Freund's adjuvant for a booster vaccine.

### Example 2

### Layer Bird Vaccination Trial

To determine the effectiveness of a vaccine containing CS-FUM-KLH and CS-DON-KLH prepared in Example 1, vaccine trials were performed with layer birds.

Two doses of 0.1 mL of 100 ng CS-FUM-DON-KLH+Freund's adjuvant were administered to layer birds 7 days apart. Serum was collected pre-booster, and six and seven days post-booster vaccination. The presence of anti-FUM and anti-DON antibodies was determined by ELISA.

As seen in FIG. 3, anti-FUM IgY and anti-DON IgY were detected at 6 and 7 days post-vaccination in layer birds fed regular diet. Negative control (non-vaccinated animals) is shown as a black bar; anti-FUM IgY is shown as a striped bar; anti-DON IgY is shown as a gray bar. The Y axis shows the absorbance value at 450 nm. The X axis shows the collection days. This nanoparticle vaccine showed sustained antibody levels (“depot” effect).

Furthermore, the nanoparticle (CS-FUM-KLH and CS-DON-KLH) vaccine did not cause adverse effects on the treated chickens. There was a 0% mortality; no detectable swelling at the injection site; and egg production was similar to the one prior to vaccination. To determine if vaccination with CS-FUM-KLH and CS-DON-KLH caused weight loss, the weight of the birds was measured prior to vaccination and 7 days post vaccination. As seen in FIG. 4, the weight of the birds prior to vaccination was slightly lower than the weight of birds 7 days post vaccination.

This Example shows that a single dose of a CS-FUM-KLH and CS-DON-KLH vaccine successfully induced anti-FUM and anti-DON antibodies, and that a booster dose increased these antibodies in vivo. The Example also shows that the CS-FUM-KLH and CS-DON-KLH vaccine showed “depot” effect (sustained antibody levels).

### Example 3

### Broiler Bird Vaccination Trials

Broiler birds were vaccinated with CS-FUM-KLH and CS-DON-KLH on day 0 followed by a booster on day 7. Production performance data was gathered on days 7, 14, and 21, and serum was collected on days 14 and 21. Bile, gut integrity, jejunal histology, jejunal tight junction protein analysis, liver histology, blood chemistry, recall assay, and serum enzyme activity were determined on day 21.

One hundred ng CS-FUM-DON-KLH in 1 mL PBS (pH 7.2) was mixed with 1:1 with complete Freund's adjuvant. Six-layer chickens were immunized intra-muscularly with 100 μL of 100 ng of CS-FUM-DON-KLH+Freund's adjuvant. A booster dose was administered 7 days post vaccination by injecting 100 ng CS-FUM-KLH and CS-DON-KLH in 1 mL PBS (pH 7.2) mixed with 1:1 with incomplete Freund's adjuvant intra-muscularly. Pre-vaccination serum was collected as control. Pre-vaccination and post-vaccination serum was collected to analyze for anti-FUM and anti-DON antibodies in the serum.

A total of 144-one-day old broiler birds were randomly allocated to four treatment groups as described in Table 3 below.

TABLE 3
Treatment Groups

| Treatment | |
|---|---|
| number | Treatment |
| 1 | Control |
| 2 | FUM + DON (feed) |
| 3 | CS-FUM-DON-KLH vaccine |
| 4 | FUM + DON (feed) |
| | FUM-DON-KLH vaccine |

Each treatment was replicated in 6 pens with 6 birds per replicate (n=6). Birds in the control group received a basal diet and were not vaccinated. Birds in the FUM+DON (feed) group received basal diet mixed with 16.4 ppm FUM and 20.9 ppm DON. Birds in vaccinated group received two doses of vaccine and were vaccinated i/m with 50 ng of FUM-KLH+50 ng of DON-KLH on day 1 and day 7. On day 21 the following parameters were measured. Production performances, serum and bile anti-FUM/DON IgY content were measured following Shanmugasundaram, R. et al. (2023, “Subclinical doses of dietary fumonisins and deoxynivalenol cause cecal microbiota dysbiosis in broiler chickens challenged with Clostridium perfringens,” Front. Microbiol. 14), gut integrity analysis and jejunal tight junction protein analysis were performed following Shanmugasundaram, R., et al. (2022, “Subclinical doses of combined fumonisins and deoxynivalenol predispose Clostridium perfringens-inoculated broilers to necrotic enteritis,” Front. Physiol. 13: 934660), Jejunal villi height and crypt depth were measured following Shanmugasundaram R., et al. (2020, “Research Note: Effect of Synbiotic Supplementation on Caecal Clostridium perfringens Load in Broiler Chickens with Different Necrotic Enteritis Challenge Models. Poult. Sci. 99 (5): 2452-2458), liver histology was performed following Yildirim, E., et al. (2011, “Effects of yeast glucomannan on performance, some biochemical parameters and pathological changes in experimental aflatoxicosis in broiler chickens,” Rev. Med. Vet. 162: 413-420), The blood samples were collected from the brachial vein without anticoagulant. The serum was analyzed for phosphorus, total protein (TP), glucose (GLU), potassium (K+), calcium (Ca2+), uric acid (UA), creatine kinase (CK), and aspartate aminotransferase (AST) using Vetscan® VS2 Chemistry Analyzer (Abaxis, Inc., Union City, CA), and FUM/DON-antigen specific cell numbers were calculated using recall assay (Zhao, K., et al., 2012, “Preparation and efficacy of a live Newcastle disease virus vaccine encapsulated in chitosan nanoparticles, PLOS ONE 7(12), e53314).

On d21, gut integrity was measured using the fluorescein isothiocyanate dextran (FITC-d; molecular weight 4000 dalton; Sigma-Aldrich) as described by Shanmugasundaram R. et al., 2022, supra). One bird/pen (n=6) was orally gavaged with 1 mL of 2.2 mg/mL FITC-d. After 2 hours, the birds were euthanized, and 3 mL of blood was collected by cardiac puncture. Blood samples were centrifuged at 450×g for 10 minutes to collect serum. Duplicate measurements were conducted on each sample on a black opaque flat bottomed 96 well plate, and the serum FITC-d concentration was determined based on a standard curve. A standard curve with 0, 0.2, 0.4, 0.6, 0.8, 1.0, and 2 g/mL FITC-d was drawn using Gen5 software on the same plate as the samples. The fluorescence levels in the serum and the standards were measured at an excitation wavelength of 485 nm and an emission wavelength of 528 nm (Synergy HT, multi-mode microplate reader, BioTek Instruments, Inc., Winooski, VT, USA). The FITC-d concentration per mL of serum was calculated based on the standard curve and reported as ng/ml.

On d21, one bird per pen (n=6) was euthanized and portion of distal-jejunum and proximal ileum (1 cm proximal and 1 cm distal to the Meckel's diverticulum) were collected in cryovials containing RNAlater® (Ambion Inc., Austin, TX, USA) and stored at −70° C. until further analysis. The jejunum was analyzed for Occluden, Zona-Occluden-1, Claudin-1, Claudin-2, and Claudin-4 tight junction protein mRNAs, were analyzed by real-time PCR as described previously (Shanmugasundaram R. et al., 2022, “Subclinical doses of combined fumonisins and deoxynivalenol predispose Clostridium perfringens-inoculated broilers to necrotic enteritis,” Front. Physiol. 13, p. 934660). Briefly, total RNA was extracted from all experimental groups using the TRI reagent (Molecular Research Center; Cincinnati, OH, USA) following the manufacturer's instructions. RNA concentration and purity were determined by Epoch spectrophotometer (BioTek Instruments Inc.), using the 260/280 and 260/230 ratios. Two mg RNA was reverse-transcribed into cDNA using high-capacity cDNA synthesis kits (Applied Biosystems, Foster City, CA, USA). The jejunal cDNA was analyzed for Occludin, Zona-Occluden-1, Claudin-1, Claudin-2, and Claudin-4 by real-time PCR (CFX96 Touch Real-Time System, BioRad; Hercules, CA, USA) using SYBR® Green PCR Master Mix (BioRad). Primer sequences and annealing temperature are provided in Table 4 below.

TABLE 4
Primer Sequences and Annealing Temperatures

| | Primer Sequence1 | Temp | | |
|---|---|---|---|---|
| Gene | (5'-3') | (C) | Type | Accession number |
| RPS-13 | CAAGAAGGCTGTTGCTGTTCG | 55 | F | NM_001001783.2 |
| | GGCAGAAGCTGTCGATGATT | | T | |
| Claudin-1 | CATACTCCTGGGTCTGGTTGGT | 55 | F | AY750897.1 |
| | GACAGCCATCCGCATCTTCT | | R | |
| Claudin-2 | CCTGCTCACCCTCATTGGAG | 55 | F | NM_001277622.1 |
| | GCTGAACTCACTCTTGGGCT | | R | |
| Zona Occluden-1 | TGTAGCCACAGCAAGAGGTG | 56 | F | XM_413773.4 |
| | CTGGAATGGCTCCTTGTGGT | | R | |
| Occludin | CCGTAACCCCGAGTTGGAT | 55 | F | NM_205128.1 |
| | ATTGAGGCGGTCGTTGATG | | R | |
| Claudin-4 | GAAGCGCTGAACCGATACCA | 56 | F | AY435420.1 |
| | TGCTTCTGTGCCTCAGTTTCC | | R | |
| GAPDH | CCTCTCTGGCAAAGTCCAAG | SS | F | NM_204305.1 |
| | GGTCACGCTCCTGGAAGATA | | R | |
| β-actin | GACTGCTGCTGACACCTTCA | 57.5 | F | NM_001303173.1 |
| | ACCGGACTGTTACCAACACC | | R | |

Each well contained 10 μl SYBR® Green PCR master mix, 7 μL RNAse-free water, 2 μL (~600 ng/μL) cDNA, 0.5 μL forward primer (5 μM), and 0.5 μL reverse primer (5 μM). To perform real-time PCR, the following settings were used for all genes: an initial denaturation of 95° C. for 10 minutes (1 cycle), followed by 95° C. for 15 seconds, and 60° C. for 45 seconds (40 cycles). The melting profile was determined by heating samples at 65° C. for 30 seconds and then increasing the temperature at a linear rate of 10° C./second to 95° C. while continuously monitoring fluorescence. Housekeeping genes of β-actin, glyceraldehyde-3-phosphate dehydrogenase (GAPDH), and Ribosomal protein S13 (RPS13) were selected, and the stability was analyzed using Normfinder software (Department of Molecular Medicine, Aarhus University Hospital, Denmark) as described previously (Shanmugasundaram R et al., 2018, “Effect of embryonic thermal manipulation on heat shock protein 70 expression and immune system development in Pekin duck embryos,” Poult. Sci. 97(12) 4200-4210). The RPS13 gene was selected for data normalization because it was the most stable expression among the set of housekeeping genes analyzed for normalization. The 2^{~}ΔΔCt method previously described by Livak K J and Schmittgen T D (2001, “Analysis of relative gene expression data using real-time quantitative PCR and the 2(−Delta Delta C(T)) Method,” Methods 25(4): 402-408), where Ct is the threshold cycle, was used to calculate the mRNA fold change. The fold change was calculated as 2(Ct Sample−housekeeping)/2(Ct Reference−housekeeping). The reference group was the Control group.

On d21, liver samples and jejunal samples from each treatment group were fixed in a 10% buffered formalin solution and processed using a tissue processor as described earlier (Shanmugasundaram R et al., 2023, supra). Briefly, samples were processed at room temperature in a graded series of alcohols (30 minutes each in 50%, 70%, 95% ethanol, and 100% ethanol with one change at 30 minutes). Samples were cleared using Pro-par (Anatech, Battle Creek, MI, USA) for 45 minutes with 2 changes at 30 minutes, followed by paraffin infiltration at 60° C. overnight with one change at 30 minutes using a tissue processor (Sakura Finetek USA, Inc., Torrance, CA, USA). Then the samples were embedded in paraffin blocks. The paraffin blocks were cut into 5-μm cross-sections and mounted on SUPERFROST® slides (Thermo Fisher Scientific, Waltham, MA, USA), stained with hematoxylin and eosin. A total of 10 fields/treatment were randomly observed under an Olympus BX60 brightfield microscope (Olympus Corp., Tokyo, Japan), and changes in the liver histopathological parameters, including fibrosis in the portal area, bile duct proliferation, hepatocyte degeneration, and cellular infiltration, were observed under 20× magnification and scored as no changes (−), mild (+), moderate (++), and severe (+++), respectively, (Yildirim et al., 2011, supra). Jejunal cross-sections were viewed under an Olympus BX60 brightfield microscope, using the CELLSENS Imaging software (Olympus America, Central Valley, PA, USA) to measure villi length and crypt depth. Ten intact lamina propria villi and crypts per section and 5 sections per sample were analyzed as described earlier (Shanmugasundaram et al, 2020, supra). The tip of the villus to the villus-crypt junction was measured as villus height. The crypt depth was defined by depth of the invagination between adjacent villi.

As seen in FIG. 5A and FIG. 5B, no anti-FUM IgY or anti-DON IgY was detectable in non-vaccinated animals fed FUM+DON, while they were detected in both, vaccinated animals fed regular diet and vaccinated animals fed a diet supplemented with FUM+DON.

Twenty one days post vaccination, the weight of non-vaccinated birds fed regular diet was the same as that of vaccinated birds fed regular diet. As seen on FIG. 6, non-vaccinated birds fed diet supplemented with FUM and DON had lost an average of 80.2 grams per bird, and vaccinated birds that were fed a diet supplemented with FUM and DON gained more weight than vaccinated or non-vaccinated birds fed regular diet.

The feed consumption of vaccinated and non-vaccinated birds was also measured. The presence of FUM and DON in the feed appeared to improve appetite, as was vaccination with CS-FUM-DON-KLH. As seen in FIG. 7, the food consumption of vaccinated birds feed a diet supplemented with FUM+DON was highest, with vaccinated birds fed a regular diet next, non-vaccinated birds feed a diet supplemented with FUM and DON next, and non-vaccinated birds fed a non-supplemented diet eating the least. As seen on FIG. 8, mycotoxins worsened feed conversion rate (FCR) by body weight by 19 points. This effect was reversed in birds vaccinated with CS-FUM-DON-KLH fed a regular diet or fed a diet supplemented with FUM and DON.

In poultry research, serum fluorescein isothiocyanate dextran (FITC-d) is a non-digestible, fluorescently labeled molecule used to measure intestinal permeability. FITC-d is too large to pass through the intestinal barrier under normal conditions, but during times of inflammation, stress, or infection, it can move from the lumen into the bloodstream through passive paracellular transport. The presence of FITC-d in serum can indicate inflammation and disruption of tight junctions. Lower serum concentrations of FITC-d indicate better intestinal integrity. As shown in FIG. 9, mycotoxins increased serum FITC-d by body weight by 1.03 g/mL as measured at day 21. The CS-FUM-DON-KLH vaccine reversed the mycotoxin-induced loss in gut integrity in birds fed with regular diet, or fed regular diet supplemented with FUM and DON.

As seen in FIG. 10A, DON, being a chemical did not induce anti-DON antibodies in bile collected at day 21 from birds exposed to DON through feed. When linked with CS and KLH, DON induced anti-DON antibodies in vaccinated birds fed a regular diet. Exposure to DON through feed further increased the anti-DON antibodies in vaccinated birds. Similarly, as seen in FIG. 10B, FUM, being a chemical did not induce anti-FUM antibodies in bile collected at day 21 from birds exposed to FUM through feed. When linked with CS and KLH, FUM induced anti-FUM antibodies in vaccinated birds fed a regular diet. Exposure to FUM through feed further increased the anti-FUM antibodies in vaccinated birds.

Similar results were obtained for serum collected at day 21. As seen in FIG. 11A, DON, being a chemical did not induce anti-DON antibodies in serum collected at day 21 from birds exposed to DON through feed. When linked with CS and KLH, DON induced anti-DON antibodies in vaccinated birds fed a regular diet. Exposure to DON through feed further increased the anti-DON antibodies in vaccinated birds. Similarly, as seen in FIG. 11B, FUM, being a chemical did not induce anti-FUM antibodies in serum collected at day 21 from birds exposed to FUM through feed. When linked with CS and KLH, FUM induced anti-FUM antibodies in vaccinated birds fed a regular diet. Exposure to FUM through feed further increased the anti-FUM antibodies in vaccinated birds.

FUM and DON induce decrease in villi height and crypt depth at the jejunal (intestinal) villi. As seen in Table 5 below, addition of FUM and DON to the diet decreased the villi height and crypt depth, the nanoparticle vaccine (CS-FUM-DON-KLH) reversed these effects.

TABLE 5
Villi Height and Crypt Depth

| | Jejunum Villus | Crypt Depth | |
|---|---|---|---|
| Treatment | Length (μm) | (μm) | Ratio |
| Control | 897.0ª | 124.6ª | 7.4 |
| FUM + DON in feed | 687.6^{b} | 99.4^{b} | 7.4 |
| Vaccine | 893.9ª | 125.4ª | 7.2 |
| Vaccine + FUM + DON | 874ª | 128.2ª | 6.9 |
| Standard Error | 40.3 | 6.0 | 0.5 |
| P value | 0.0036 | 0.0091 | 0.9219 |

Furthermore, as shown in Table 6 below, the nanoparticle vaccine (CS-FUM-DON-KLH) maintained the tight junction protein expression during FUM and DON exposure in jejunum. Occludin is a transmembrane protein that regulates the permeability of epithelial and endothelial barriers. It was first identified in epithelial cells as a 65 kDa integral plasma-membrane protein localized at the tight junctions.

TABLE 6
Effect on Protein Expression

| | occluden | Cla-1 | Cla-2 | Cla-4 | ZO |
|---|---|---|---|---|---|
| Control | 1 | 1 | 1 | 1 | 1 |
| FUM + DON | 1.8 | −2 | 3.4 | −1.6 | −1.9 |
| in feed | | | | | |
| Vaccine | 1.6 | 1.6 | 1.1 | 1 | 1.1 |
| Vaccine + | 1.3 | 1.5 | 1.1 | 1.2 | 1.2 |
| FUM + DON | | | | | |
| Standard Error | 0.26 | 0.28 | 0.6 | 0.15 | 0.10 |
| P value | 0.1531 | 0.058 | 0.0233 | 0.0015 | 0.006 |

The nanoparticle vaccine reversed liver damage caused by FUM and DON. As seen in FIG. 12, birds fed diet supplemented with FUM and DON presented with mononuclear cell infiltration (MNCI), fibrosis and bile duct proliferation (F+BDP), and hydroscopic hepatocyte degeneration (HHD), while control birds, and birds receiving the nanoparticle vaccine presented with normal liver.

The recall assay is an assay used for quantifying antigen-specific memory T-cells. When exposed to antigen in vitro, antigen-specific T-cells will rapidly proliferate and stay alive. Presence of live cells (OD values in MTT assay) at three days of culture indicates the presence of antigen-specific memory lymphocytes. Mononuclear cells from spleen were collected on day 21 and stimulated in vitro with either 0 (control) or 1 g/mL CS-FUM-DON-KLH for three days. Live cells were measured using the 3-[4,5-dimethylthiazol-2-yl]-2,5-diphenyl tetrazolium bromide (MTT) colorimetric assay with absorbance measured at OD570 nm. The data in FIG. 13 shows that the groups vaccinated with the nanoparticle vaccine had slightly higher FUM and DON specific memory lymphocytes, indicating a successful vaccine.

The amount of serum aspartate aminotransferase and creatine kinase on day 21 was also measured. As seen in FIG. 14A and FIG. 14B, the levels of serum aspartate aminotransferase and creatine kinase increased in birds fed a diet supplemented with FUM and DON, while they were very similar in control and vaccinated birds. The presence of FUM and DON in the feed did not appear to make a difference.

The results of the blood chemistry analyses performed on day 21 are shown in Table 7 below.

TABLE 7
Blood Chemistry Results

| | | Total | | | Uric |
|---|---|---|---|---|---|
| | Phosphorus | Protein | Glucose | Albumin | Acid |
| | (mg/dL) | (g/dL) | (mg/dL) | (g/dL) | (mg/dL) |
| Control | 9.3 | 2.9ª | 253.5 | 2.3 | 8.0 |
| FUM + DON in feed | 7.9 | 1.7^{b} | 198.0 | 2.1 | 5.8 |
| Vaccine | 8.4 | 3.1ª | 238.7 | 2.1 | 5.0 |
| Vaccine + FUM + | 8.7 | 3.1ª | 248.0 | 2.4 | 7.1 |
| DON | | | | | |
| Standard Error | 0.6 | 0.3 | 20.5 | 0.1 | 1.9 |
| P value | 0.4 | 0.0046 | 0.2 | 0.3 | 0.7 |

These results show that the nanoparticle vaccine reversed FUM and DON-induced loss in body weight, increase in FCR, disruption of gut integrity, decrease in jejunal villi height and crypt depth, liver damage, and increase in serum aspartate aminotransferase and creating kinase.

### Example 4

### Anti-FUM and Anti-DON Antibodies in Egg Yolks

To determine if layer birds transfer the “neutralizing” antibodies to egg yolk, the amounts of IgG Y anti-FUM and anti-DON in birds vaccinated with CS-FUM-KLH and CS-DON-KLH were measured using ELISA.

Egg yolk antibodies were extracted following Goldring, J. D. and Coetzer, T. H. (2003, “Isolation of chicken immunoglobulins (IgY) from egg yolk,” Biochem. Mol. Biol. Educ. 31(3): 185-187). The egg yolks were separated from the egg whites and excess white was removed by washing the egg yolks with PBS. The yolk sac was punctured, and the yolk was collected. The yolk volume was measured using a measuring cylinder. Two volumes of PBS (pH7.6) containing 0.02% (W/V) sodium azide was added to the yolk and mixed using a glass rod. Polyethylene glycol (PEG) 6000 was added at 3.5% (W/V). The sample was centrifuged at 4000×g for 30 minutes at room temperature and the supernatant was collected. The supernatant was filtered in a funnel plugged with fiberglass wool to trap the lipid fraction. PEG 6000 was added at 8.5% (W/V) to the filtrate. The solution was centrifuged at 10,000×g for 15 minutes. The supernatant was discarded, and the pellet was resuspended in a volume of PBS equal to the egg yolk volume. PEG 6000 was added at 12% (W/V), mixed, and centrifuged at 10,000×g for 15 minutes. The supernatant was discarded, and the final pellet was dissolved in PBS in one fourth of the original egg yolk volume. The samples were stored at 4° C. The total protein (IgY) concentration was quantified using a NANODROP A280 reader on a Synergy HTX spectrophotometer (Biotek; Santa Clara, CA, USA), and the concentration was reported in mg/mL. The IgY specificity against FUM and DON was measured using ELISA as described above.

Table 8 below shows preliminary ELISA data of quantification of anti-DON antibodies in egg yolk.

TABLE 8
Egg Yolk Anti-DON Antibodies

| Negative Ctrl. | | Sample 1 | | Sample 2 | | |
|---|---|---|---|---|---|---|
| 7 | 8 | 9 | 10 | 11 | 12 | Dilution |
| 0.002 | 0.001 | 0.095 | 0.101 | 0.208 | 0.196 | 1:10 |
| −0.001 | −0.001 | 0.025 | 0.03 | 0.027 | 0.036 | 1:100 |
| −0.002 | −0.002 | 0.004 | 0.005 | 0.005 | 0.006 | 1:200 |
| −0.001 | −0.002 | 0 | 0.003 | 0 | 0.004 | 1:500 |
| 0.001 | −0.002 | 0.008 | −0.001 | −0.001 | 0.001 | 1:1000 |
| −0.001 | −0.001 | −0.003 | −0.003 | −0.002 | 0.001 | Blank |

The results obtained by quantifying anti-FUM antibodies using ELISA 5 days after booster are shown below in Tables 9A and 9B below.

TABLE 9A
Anti-FUM detected in yolks

| | Negative | | Sample 1 | | Sample 2 | |
|---|---|---|---|---|---|---|
| Dilution | 1 | 2 | 3 | 4 | 5 | 6 |
| 1:1 | 0.002 | 0.001 | 0.295 | 0.295 | 0.306 | 0.304 |
| 1:10 | 0 | 0 | 0.138 | 0.135 | 0.164 | 0.142 |
| 1:100 | −0.002 | 0.002 | 0.027 | 0.027 | 0.035 | 0.032 |
| 1:200 | 0 | 0.001 | 0.012 | 0.013 | 0.011 | 0.012 |
| 1:500 | −0.002 | −0.002 | 0.008 | 0.009 | 0.008 | 0.008 |
| 1:1000 | −0.001 | 0 | 0.005 | 0.006 | 0.005 | 0.004 |
| Blank | −0.001 | −0.001 | −0.002 | −0.002 | −0.003 | −0.003 |

TABLE 9B
Anti-FUM detected in yolks

| | Negative | | Sample 3 | | Sample 4 | |
|---|---|---|---|---|---|---|
| Dilution | 7 | 8 | 9 | 10 | 11 | 12 |
| 1:1 | 0.005 | 0.012 | 0.293 | 0.303 | 0.381 | 0.404 |
| 1:10 | 0 | 0.002 | 0.186 | 0.193 | 0.25 | 0.289 |
| 1:100 | 0.004 | 0.002 | 0.044 | 0.044 | 0.091 | 0.098 |
| 1:200 | 0.001 | 0 | 0.017 | 0.024 | 0.054 | 0.053 |
| 1:500 | −0.001 | 0.003 | 0.01 | 0.019 | 0.021 | 0.032 |
| 1:1000 | −0.001 | −0.001 | 0.005 | 0.008 | 0.016 | 0.079 |
| Blank | −0.002 | −0.001 | 0.001 | 0.002 | 0.003 | 0.007 |

As seen in FIG. 15, no IgY was detected in serum from chickens not vaccinated with CS-FUM-KLH and CS-DON-KLH, while anti-FUM IgY was detected in serum from vaccinated chickens at all timepoints measured, with a peak around 6 dpv, and anti-DON IgY was also detected in serum from vaccinated chickens at all timepoints measured, with a peak around 7 dpv.

Detection of IgY in egg yolks presented with a similar pattern. As seen in FIG. 16, no IgY was detected in eggs from chickens not vaccinated with CS-FUM-KLH and CS-DON-KLH, while anti-FUM IgY and anti-DON IgY were detected in eggs from vaccinated chickens at all timepoints measured, with a peak around 6 dpv.

Four different samples were assayed using ELISA to determine the lowest dilution of egg yolk powder that recognizes FUM in egg yolks from eggs laid 5 days post-booster vaccination. As seen in FIG. 17, FUM was detected at an egg yolk concentration of about 9 μg/mL to about 10 μg/mL.

The data in this Example shows that anti-FUM and anti-DON IgY was detected in chicken serum and yolks from eggs laid by chickens vaccinated with CS-FUM-KLH and CS-DON-KLH,

### Example 5

### In Vitro Studies

The in vitro effect of FUM, DON, and/or egg yolk powder from CS-FUM-KLH and CS-DON-KLH-vaccinated chickens were tested on the chicken macrophage cell line, HD11 cells.

To prepare egg yolk powder, the egg yolks from control and vaccinated chicken were freeze dried and shipped to a commercial granulator for granulation using fluid bed drying. The granulated egg powder mixed at 0.05% in the experimental diet for in vivo experiments.

One×10⁶ HD11 cells were plated per well of 96 well plates in complete Iscove's Modified Dulbecco's Medium (IMEM) containing 4% Fetal bovine serum, 2% chicken serum, and 1.0% penicillin and streptomycin, and incubated at 42° C. until confluence. The following eight experimental groups were repeated in duplicates with 6 replicates (n=6). 1. Cells were incubated in media only (Control); 2. Cells were incubated in media supplemented with 50 μg/mL FUM (FUM); 3. Cells were incubated in media supplemented with 10 μg/mL DON (DON); 4. Cells were incubated in media supplemented with 50 μg/mL FUM and 10 μg/mL DON (FUM+DON); 5. Cells were incubated in media supplemented with 10 μg/mL egg yolk powder from CS-FUM-KLH and CS-DON-KLH-vaccinated chickens (Control+AB); 6. Cells were incubated in media supplemented with 50 μg/mL FUM and 10 μg/mL egg yolk powder from CS-FUM-KLH and CS-DON-KLH-vaccinated chickens (FUM+AB); 7. Cells were incubated in media supplemented with 10 μg/mL DON and 10 μg/mL egg yolk powder from CS-FUM-KLH and CS-DON-KLH-vaccinated chickens (DON+AB); and 8. Cells were incubated in media supplemented with 50 μg/mL FUM, 10 μg/mL DON, and 10 μg/mL egg yolk powder from CS-FUM-KLH and CS-DON-KLH-vaccinated chickens (FUM+DON+AB).

Cells were incubated for 48 hours. The effects of FUM, DON, and/or egg yolk antibodies were measured by quantifying apoptotic cells in a flow cytometer and measuring nitric oxide production by colorimetry assay (Shanmugasundaram R, et al, 2019, “Effect of embryonic thermal manipulation on heat shock protein 70 (HSP70) expression and subsequent immune response to post-hatch lipopolysaccharide challenge in Pekin ducklings,” Poultry Sci. 98(2):722-733). This experiment was repeated three times.

The data in FIG. 18A shows that FUM induces apoptosis in 30% of HD11 cells, and FIG. 18B shows that addition of egg yolk powder obtained from eggs of anti-mycotoxin-vaccinated chickens reversed the FUM effect on the HD11 cells. Similarly, FIG. 18C shows that DON induces apoptosis in 50% of HD11 cells, and FIG. 18D shows that addition of egg yolk powder obtained from eggs of anti-mycotoxin-vaccinated chickens reversed the DON effect on the HD11 cells. The data in FIG. 19 shows that addition of FUM, DON, or FUM and DON induce early and late HD11 cell apoptosis, but these effects are reversed when egg yolk powder from eggs laid by anti-FUM/DON vaccinated chickens.

As seen on FIG. 20, FUM decreases NO production by macrophage cell lines as expected, but addition of egg yolk powder from eggs laid by anti-FUM/DON vaccinated chickens reverses this effect.

### Example 6

### Broiler Bird Trials

Broiler birds were fed vaccinated with CS-FUM-KLH and CS-DON-KLH on day 0 followed by a booster on day 7. Production performance data was gathered on days 7, 14, and 21, and serum was collected on days 14 and 21. Bile, gut integrity, jejunal histology, jejunal tight junction protein analysis, liver histology, blood chemistry, recall assay, and serum enzyme activity were determined on day 21.

A total of 144-day old broiler birds were randomly allotted to four treatment groups: 1. Fed regular diet only (Control); 2. Fed diet supplemented with 16.4 ppm FUM and 20.9 ppm DON (FUM+DON); 3. Fed diet supplemented with 0.05% (500 g/1000 Kg of feed) egg yolk powder from CS-FUM-KLH and CS-DON-KLH-vaccinated chickens. 4. Fed diet supplemented with 16.4 mg FUM/Kg diet, 20.9 mg DON/Kg diet, and 0.05% Egg Yolk Powder.

Each treatment was replicated in 6 pens with 6 birds per replicate (n=6). On day 21 the following parameters were measured. Production performances, serum and bile anti-FUM/DON IgY content, gut integrity analysis, Jejunal villi height and crypt depth, jejunal tight junction protein analysis, liver histology, blood chemistry, serum Aspartate Aminotransferase and Creatinine Kinase concentration, and FUM/DON-antigen specific cell numbers using recall assay, following the same procedures as described in Example 3, above.

As seen in FIG. 21, mycotoxins decreased body weight at d21 by 80.2 g per bird. Addition of egg yolk powder obtained from eggs of CS-FUM-DON-KLH-vaccinated chickens reversed this effect (student t test; P=0.10). FIG. 22 shows that addition of FUM and DON numerically increased feed consumption at day 21, and this effect was not reversed when the chickens were fed egg yolk powder obtained from eggs of CS-FUM-DON-KLH-vaccinated chickens. The data in FIG. 23 shows that mycotoxins worsened FCR by body weight by 19 points, but this effect was reversed when the chickens were fed a diet supplemented with egg yolk powder obtained from eggs of CS-FUM-DON-KLH-vaccinated chickens.

As seen in FIG. 24, the liver from chickens fed diet supplemented with mycotoxins appeared lighter than those from chickens fed regular diet, diet supplemented with egg yolk powder obtained from eggs of CS-FUM-DON-KLH-vaccinated chickens, or fed diet supplemented with FUM, DON, and egg yolk powder obtained from eggs of CS-FUM-DON-KLH-vaccinated chickens.

FIG. 25 shows that addition of mycotoxins to the feed increased serum FITC-d by body weight by 1.03 μg/mL, while addition of egg yolk powder obtained from eggs of CS-FUM-DON-KLH-vaccinated chickens to the feed, either alone or in the presence of mycotoxins, reversed this effect.

Because egg yolk powder acts by neutralizing the mycotoxins in the gut, as seen in FIG. 26, as expected, adding egg yolk powder obtained from eggs of CS-FUM-DON-KLH-vaccinated chickens to the feed did not increase the anti-FUM bile IgA.

The concentration of FUM and DON in feed was measured by high performance liquid chromatography (HPLC). As seen in Table 10A and Table 10B below, feed was way above the target concentration of 3 ppm for FUM and 4 ppm for DON.

TABLE 10A

| Treatment | AFB1 | AFB2 | Tot. AFL | FB1 | FB2 | FB3 | Tot. FB |
|---|---|---|---|---|---|---|---|
| Description | (ppm) | (ppm) | (ppm) | (ppm) | (ppm) | (ppm) | (ppm) |
| Ctrl. diet | 0.008 | <LOD* | 0.008 | 1.501 | 0.5178 | <LOQ* | 2.0987 |
| MTX diet | 0.0015 | <LOD | 0.0015 | 15.7308 | 0.5501 | 0.1871 | 16.468 |
| Ctrl + IgY | 0.0001 | <LOD | 0.0001 | 1.4008 | 0.4245 | <LOQ | 1.9053 |
| MTX + IgY | 0.00019 | <LOD | 0.00019 | 15.7001 | 0.5213 | 0.194 | 16.4514 |

TABLE 10B

| | | 15 acyl | Type B | T-2 | | Type A | |
|---|---|---|---|---|---|---|---|
| Treatment | DON | DON | trich | toxin | HT-2 | trich | ZEA |
| Description | (ppm) | (ppm) | (ppm) | (ppm) | (ppm) | (ppm) | (ppm) |
| Ctrl. diet | 1.4587 | <LOD | 1.4587 | <LOD | <LOD | <LOD | 0.0883 |
| MTX diet | 20.9632 | <LOD | 21.1382 | <LOD | <LOD | <LOD | 1.9188 |
| Ctrl + IgY | 1.6665 | <LOD | 1.6665 | <LOD | <LOD | <LOD | 0.054 |
| MTX + IgY | 19.8964 | 0.4067 | 20.3031 | <LOD | <LOD | <LOD | 2.2748 |

LOD=limit of detection; LOQ=limit of quantitation; AFB1=aflatoxin B1; AFB2=aflatoxin B2; AFL=aflatoxin; FB1=fumonisin B1; FB2=fumonisin B2; FB3=fumonisin B3; FB=fumonisins; DON=deoxynivalenol; 15-acetyl DON=15 acetyl deoxynivalenol; Type B Trich=Total Type B trichothecenes; Type A Trich—Total Type A trichothecenes.

As seen in the images presented in FIG. 27, addition of FUM and DON to the chicken feed induced decrease in villi height and crypt depth at the jejunal (intestinal) villi. This effect was reversed by the addition to the feed of 0.005% egg yolk powder obtained from eggs of CS-FUM-DON-KLH-vaccinated chickens. The data in Table 11 below, shows that supplementation with 0.05% egg powder reversed the induced loss in villi height and crypt depth-induced by the addition of FUM and DON to the feed.

TABLE 11

| | Jejunum | Crypt | Villus |
|---|---|---|---|
| | Villus | depth | length/crypt |
| Treatment | length (μm) | (μm) | depth ratio |
| Control | 897.0ª | 124.6ª | 7.4 |
| FUM + DON | 687.6^{b} | 99.4^{b} | 7.1 |
| Egg powder (IgY) | 947.2ª | 138.2ª | 6.9 |
| Egg powder + | 977.0ª | 133.6ª | 7.4 |
| FUM + DON | | | |
| SE | 41.9 | 6.3 | 0.5 |
| P value | 0.0004 | 0.0014 | 0.8996 |

FIG. 28 depicts images of Liver sections from layer birds fed regular diet (Control), fed a diet supplemented with FUM and DON (FUM+DON), fed a diet supplemented with egg yolk powder containing anti-FUM and anti-DON IgY (Egg Powder), or fed a diet supplemented with FUM and DON and egg yolk powder containing anti-FUM and anti-DON IgY (FUM+DON+Egg Powder). The mononuclear cell infiltration (MNCI), fibrosis and bile duct proliferation (F+BDP), and the hydropic degeneration (HD) are indicated by arrows in the image of the liver section from chickens fed a diet supplemented with FUM and DON. These damages were reversed in chickens fed diets supplemented with egg yolk powder containing anti-FUM and anti-DON IgY.

Table 12 below shows that supplementation with egg yolk powder maintained the tight junction protein expression during jejunum FUM and DON exposure.

TABLE 12

| | Occluden | Cla-1 | Cla-2 | Cla-4 | ZO |
|---|---|---|---|---|---|
| Control | 1.0 | 1.0 | 1.0^{b} | 1.0 | 1.0ª |
| FUM + DON | 1.8 | −2 | 3.4ª | −1.6 | −1.9^{b} |
| Egg Powder | 1.2 | 1.0 | 1.1b | 1.8 | 1.0ª |
| FUM + DON + | 1.2 | 1.3 | 0.7b | 1.6 | 1.0ª |
| egg powder | | | | | |
| SE | 0.2 | 0.22 | 0.6 | 0.54 | 0.09 |
| P value | 0.0582 | 0.182 | 0.0143 | 047 | 0.0054 |

Table 13 below shows the results for the blood chemistry on chickens submitted to different treatments.

TABLE 13

| | | Total | | | Uric |
|---|---|---|---|---|---|
| | Phosphorous | Protein | Glucose | Albumin | Acid |
| | (mg/dL) | (g/dL) | (mg/dL) | (g/dL) | (mg/dL) |
| Control | 9.3 | 2.9ª | 253.5 | 2.3 | 8.0 |
| FUM + DON | 8.4 | 1.7^{b} | 198.0 | 2.1 | 5.8 |
| Egg Powder | 9.7 | 2.7ª | 255.7 | 2.3 | 7.3 |
| FUM + DON + | 10.6 | 2.8ª | 238.5 | 2.2 | 7.7 |
| egg powder | | | | | |
| SE | 0.6 | 0.23 | 19.7 | 01 | 1.8 |
| P value | 0.070 | 0.004 | 0.172 | 0.630 | 0.816 |

Graphs depicting the amounts of serum aspartate aminotransferase and creatine kinase detected in layer chickens at day 21 after different treatments are shown in FIG. 29A and FIG. 29B. These graphs show that addition of only FUM and DON to the feed increases the amounts of aspartate aminotransferase and creatine kinase, while addition of egg yolk powder containing anti-FUM and anti-DON IgY reverses these effects.

We claim:

1. A composition comprising an egg or a part thereof enriched in at least one anti-mycotoxin antibody.

2. The composition of claim 1, wherein the egg or part thereof is from an anti-mycotoxin-vaccinated egg-producing animal.

3. The composition of claim 1, wherein the at least one anti-mycotoxin antibody in the composition neutralizes aflatoxin, ochratoxin, fumonisin, zearalenone, or trichothecene.

4. A feed supplement comprising the composition of claim 1.

5. The feed supplement of claim 4; for administration to a fish, a bird, a reptile, or a mammal.

6. The feed supplement of claim 5, wherein the bird is a domesticated or wild fowl bird.

7. The feed supplement claim 5, wherein the mammal is a wild or domesticated mammal.

8. A feed comprising feed material and the feed supplement of claim 5.

9. The feed of claim 8, wherein the feed comprises from at least about 0.0001% to at least about 10.00% by weight of the feed supplement.

10. The feed of claim 8, further comprising at least one additive.

11. The feed of claim 8; for administration to a fish, a bird, a reptile, or a mammal.

12. The feed of claim 11, wherein the bird is a domesticated or wild fowl bird.

13. The feed of claim 11, wherein the mammal is a wild or domesticated mammal.

14. The feed of claim 8, wherein the feed is in the form of powder, granules, pellets, tablets, capsules, gel, paste, emulsion, liquid, chew bars, chew bones, chew rolls, or treats.

15. A method for preparing a composition comprising egg or a part thereof enriched in at least one anti-mycotoxin antibody, the method comprising:
collecting eggs laid by an egg-producing animal vaccinated with at least one anti-mycotoxin vaccine;
optionally separating egg yolks or egg whites from the collected eggs; and
drying the collected eggs, egg yolks, egg whites, or a mixture thereof.

16. The method of claim 15, wherein the anti-mycotoxin vaccine comprises at least one mycotoxin bound to a carrier protein and entrapped within a nanoparticle.

17. The method of claim 16, wherein the at least one mycotoxin is an aflatoxin, an ochratoxin, a fumonisin, a zearalenone, or a trichothecene.

18. The method of claim 16, wherein the carrier protein is a hemocyanin.

19. The method of claim 16, wherein the nanoparticle comprises chitosan.

20. A method of preventing or ameliorating one or more symptoms of mycotoxicosis in an animal in need thereof, the method comprising administering to the animal at least one dose of a composition comprising egg or a portion thereof enriched in at least one anti-mycotoxin antibody, wherein administering the composition to the animal protects the animal from one or more clinical signs of mycotoxin-induced mycotoxicosis.

21. The method of claim 20, wherein the composition is provided to the animal in feed or drinking water.
